SEQUENCE LISTING (10 sequences)

SEQ ID NO: 1 - DNA, 30 bp, Artificial Sequence
  Description of Artificial Sequence Synthetic primer
cggggtaccccgatgagagcgacggagatc

SEQ ID NO: 2 - DNA, 37 bp, Artificial Sequence
  Description of Artificial Sequence Synthetic primer
cgcggatccgcgttatagcaaagctctttccaagccc

SEQ ID NO: 3 - DNA, 30 bp, Artificial Sequence
  Description of Artificial Sequence Synthetic primer
ccgctcgagcggatgagagcgacggagatc

SEQ ID NO: 4 - DNA, 37 bp, Artificial Sequence
  Description of Artificial Sequence Synthetic primer
cccaagcttgggttatagcaaagctctttccaagccc

SEQ ID NO: 5 - protein, 26 aa, Artificial Sequence
  Description of Artificial Sequence Synthetic peptide
CNLEACQKRCQSLGLQGKCAGSFCAC

SEQ ID NO: 6 - DNA, 28 bp, Artificial Sequence
  Description of Artificial Sequence Synthetic primer
gcggatccgccaccatgaccatcttatg

SEQ ID NO: 7 - DNA, 35 bp, Artificial Sequence
  Description of Artificial Sequence Synthetic primer
cccaagcttgggttatcttttttctctctgcacca

SEQ ID NO: 8 - protein, 72 aa, Human immunodeficiency virus 1
PLCVTLNCTDLRNATNTTSSSWETMEKGEIKNCSFNITTSIRDKVQKEYALFYNLDVVPIDNASYRLISCNT

SEQ ID NO: 9 - protein, 53 aa, Artificial Sequence
  Description of Artificial Sequence Synthetic polypeptide
PLCVTLNCNLACQKRCQSLGLQGKCAGSFCACFYNLDVVPIDNASYRLISCNT

SEQ ID NO: 10 - protein, 87 aa, Artificial Sequence
  Description of Artificial Sequence Synthetic polypeptide
PLCVTLNCTDLRNATNTTSSSWETMEKGEIKNCSFNCNLACQKRCQSLGLQGKCAGSFCACKEYALFYNLDVVPIDNASYRLISCNT

(19) United States
(12) Patent Application Publication
(10) Pub. No.: US 2010/0303858 A1
(43) Pub. Date: Dec. 2, 2010

(54) Chimeric HIV-1 Glycoproteins and Their Biological Applications

(76) Inventors: Francisco Veas, Maugio (FR); Claudio Vita, Gif-sur-Yvette (FR); Mireille C. Sauvage, Gif-sur-Yvette (FR); Fabio Vita, Gif-sur-Yvette (FR); Elena Vita, Gif-sur-Yvette (FR); Loic Martin, Gif-sur-Yvette (FR); Dorothy Bray, Buckinghamshire (GB); Kadija Benlhassan-Chahour, Villeparisis (FR)

Correspondence Address: NIXON & VANDERHYE, PC, 901 NORTH GLEBE ROAD, 11TH FLOOR, ARLINGTON, VA, 22203, US

(21) Appl. No.: 12/085,632

(22) PCT Filed: Nov. 28, 2006
(86) PCT No.: PCT/IB2006/004037

§ 371 Date: May 11, 2010

Related U.S. Application Data
(60) Provisional application No. 60/739,936, filed on Nov. 28, 2005.

(51) Int. Cl. A61K 39/21; C07K 14/16; C07K 16/10; C12N 15/63; C12N 5/00; A61P 37/04; A61P 31/18

(52) U.S. Cl. 424/208.1; 530/350; 530/326; 530/325; 530/324; 530/389.4; 530/388.35; 435/320.1; 435/325

(57) ABSTRACT

The invention relates to chimeric HIV-1 gp120 glycoproteins, wherein at least a part of gp120 variable region V1 and/or V2 is replaced by a CD4-derived sequence to obtain the exposition of CD4 induced epitopes or CD4i capable of inducing a specific humoral immune response. Application for the preparation of vaccinal and pharmaceutical composition.

The invention relates to chimeric gp120 glycoproteins and their biological applications.

Conserved immunogens able to elicit broadly neutralizing antibodies may be an important component of an effective HIV-1 vaccine. Proteins that constitute the HIV-1 envelope, the surface unit (SU) glycoprotein (gp120) and the gp41 transmembrane (TM) glycoprotein, are potential targets to develop neutralizing prophylactic protection. Protective monoclonal antibodies, isolated from infected individuals, have revealed several neutralization epitopes on envelope glycoprotein. They include several regions in the TM gp41 and conserved structures within the SU gp120 involved in receptor binding, but also in the V2 and V3 variable loops, which are highly immunogenic, inducing only type specific antibodies. It has been difficult to generate antibodies directed against these neutralizing epitopes, however. Mainly due to a strong variability of the envelope glycoproteins and its high levels of glycosylation which can change during infection, contributing significantly to the reduction of epitope exposure, especially the receptor binding sites, to the humoral immune system.

Looking for possible interferences with HIV before cell infection, receptor binding sites and their close neighbourhoods are particularly attractive targets for HIV-1 vaccines. The gp120 envelope glycoprotein binds sequentially to the cellular receptors CD4 and a member of the chemokine receptor family, mainly CCR5 and CXCR4. Binding of the gp120 to CD4 induces conformational changes in the gp120 glycoprotein that expose the gp120 co-receptor-binding site so called CD4-induced (CD4i) epitopes. This latter is one of the most conserved surfaces on the gp120, even more than the CD4 binding site. Since co-receptor binding is one of the most important steps for subsequent virus-cell fusion and therefore crucial for HIV-1 infection, antibodies specific to CD4-induced (CD4i) epitopes on the envelope gp120 would be particularly effective to broadly block infection by different HIV-1 isolates. Several such CD4i antibodies that block co-receptor binding have been identified and characterized. One of them, termed 17b, was isolated from an HIV-1 infected individual and is capable of neutralizing some T-cell line-adapted (TCLA) HIV-1 strains, but poorly the primary isolates. E51, another CD4i binding antibody, induces a stronger neutralizing response against primary HIV-1 isolated. The limited number of these antibodies that recognize a CD4-binding conformation and neutralize primary HIV-1 isolates efficiently, show that the exposed epitopes may not be efficient to induce a specific humoral immune responses. Therefore it will be very interesting to construct a molecule capable to permanently exhibit this epitope and its neighbourhoods.

Several strategies have been employed to expose CD4i epitopes on gp120. Removal of specific N-linked glycosylation sites from variable loops of gp120 have rendered these mutant viruses more susceptible to neutralization by CD4i antibodies. Soluble crosslinked complexes of gp120 with the four extracellular domain of human CD4 have been constructed and generate neutralizing antibodies against a wide range of primary HIV-1 isolates, including antibodies with cross-reactive neutralizing activity. Although soluble proteins can prime a CD4+ T cell response, they do not efficiently induce CD8+ T cell-mediated immune response, which is known to be important to the control of HIV-1 infection. An alternative approach to overcome these problems is the construction of a single-chain polypeptide that mimic the gp120-CD4 complex intermediate. It has been constructed a HIV-1 gp120 protein linked to the first two domains of CD4 by a 20-amino-acid linker that exhibited increase exposure of CD4i epitopes, but did not mediate viral neutralization. More recently, other fusion proteins were constructed joining the JRLF gp120 protein to either the same D1 and D2 domains of CD4 or the CD4-structure derived sequence, CD4M9, which reproduce the CDR2-like loop of CD4. Only gp120-CD4_{D12} molecule induced neutralizing antibodies, but seems to be exclusively directed against CD4.

Since it was previously postulated that, the variable regions, V1/V2, of gp120 glycoprotein occludes partially the binding site for the cellular receptor and co-receptor, deletion studies of the gp120 V2 and V1/V2 loops resulted in an increased exposure of CD4i epitopes, exhibiting enhanced neutralization activity.

The inventors have found that valuable transconformed gp120-derived molecules could be obtained by replacing specific regions of gp120 by sequences derived from CD4 without using linker sequences or crosslinking agents. The research works on such chimeras shown that they are immunogenic and in combination with specific chemokine(s) form potent products for vaccinal/microbicidal applications.

It is then an object of the invention to provide chimeric HIV-1 gp120 glycoproteins.

Another object of the invention aims to provide DNA constructs coding for such molecules.

The invention also relates to the immunological and pharmaceutical applications of said chimeric gp120 proteins.

The chimeric HIV-1 gp120 glycoproteins of the invention are characterized in that at least a part of gp120 variable region V1 and/or V2 is replaced by a CD4-structure derived sequence.

The resulting chimera have similar size and structure to those of wild type gp120 and exhibit the ability to mimic most of the properties of the gp120/CD4 complex, in particular its capacity to recognize the native CCR5 receptor and to expose CD4i epitopes capable of inducing a specific humoral immune response.

In preferred chimeric glycoproteins, the CD4-derived sequence replaces V1 and a part of V2 variable regions.

Advantageously, the CD4-structure derived (sdCD4) peptide mimics the CDR2-like loop of human CD4 receptor.

Preferably, the CD4-derived peptide comprises 15 to 35 amino acids of CD4, especially 20 to 30 amino acids.

A valuable CD4-structure derived peptide has for example 28 amino acids and advantageously has sequence SEQ ID NO 5 CNLEACQKRCQSLGLQGKCAGSFCAC.

In said chimeric HIV-1 gp120 molecules, the CD4-derived sequence advantageously replaces V1 and 10 to 20 amino acids from V2 loop, for example 16 amino acids of V2 loop.

The above disclosed chimeric HIV-1 gp120 glycoproteins are further characterized by the fact that they are recognized by anti-CD4i monoclonal antibodies, but are not recognized by anti-CD4 antibodies.

The chimeric glycoproteins of the invention are then recognized by CG10, E51, and LF17 monoclonal antibodies as illustrated in the examples.

The chimeric HIV-1 gp120 glycoproteins of the invention are still characterized in that they bind CD4 receptor and CCR5 co-receptor.

The invention also relates to the immunization products, antisera and antibodies directed to CD4i epitopes exposed in the above disclosed chimeric molecules.

The antibodies comprise polyclonal antibodies such as obtained by immunization of animals and recovery from the antisera. They also comprise monoclonal antibodies such as obtained by fusion of myelomatous cells with the lymphocytes, in particular of spleen or ganglions of an animal previously immunized by injection of the chimeric molecules such as above defined, screening of the supernatants of the hybridoma obtained, for example according to ELISA or IFI techniques, so as to reveal the antibodies specifically directed against the chimeric molecules.

The hybridoma strains secreting these monoclonal antibodies are also part of the invention.

The DNA constructs encoding the above disclosed chimeric molecules are also part of the invention.

These DNA constructs are advantageously obtained by replacing in the DNA coding for a wild-type gp120 molecule, a fragment coding for V1 and/or V2 gp120 variable region with a fragment encoding a CD4-derived peptide. These fragments are advantageously obtained by a synthetic route.

The DNA constructs are then subcloned in an expression vector and used for transfection of cells.

The above disclosed chimeric HIV-1 constructs, alone or in combination, are capable of inducing a specific humoral immune response useful for preventing infections due to HIV-1 and are then powerful immunogenic tools.

The chimeric HIV-1 constructs of the invention are particularly useful with TLR ligands, for example TLR9 ligands such as CpG-like motif.

Other combinations of interest comprise said chimeric constructs with IL-22 and/or CCL28.

In particularly preferred combination such as above defined, the chimeric constructs are chimera 1 and 2 such as disclosed in the examples.

The invention thus relates to vaccine compositions specific to HIV-1 infections comprising an effective amount of at least one chimeric HIV-1 gp120 glycoproteins as above defined, with a carrier.

The formulation and the dose of said vaccine compositions can be developed and adjusted by those skilled in the art as a function of the method of administration desired, and of the patient under consideration (age, weight).

These compositions comprise one or more physiologically inert vehicles, and in particular any excipient suitable for the formulation and/or for the method of administration desired.

The invention also relates to vaccines compositions specific to HIV-1 infections further comprising a TLR ligand, 11-22 and/or CCL28 such as above defined.

The present invention is also aimed towards the use, in an effective amount, of at least one antibody such as above defined for the diagnosis of the presence or absence of HIV-1 infection.

The present application is also aimed towards any use of an antibody such as above defined for the manufacture of a composition, in particular of a pharmaceutical composition, intended to alleviate and/or to prevent and/or to treat HIV infection.

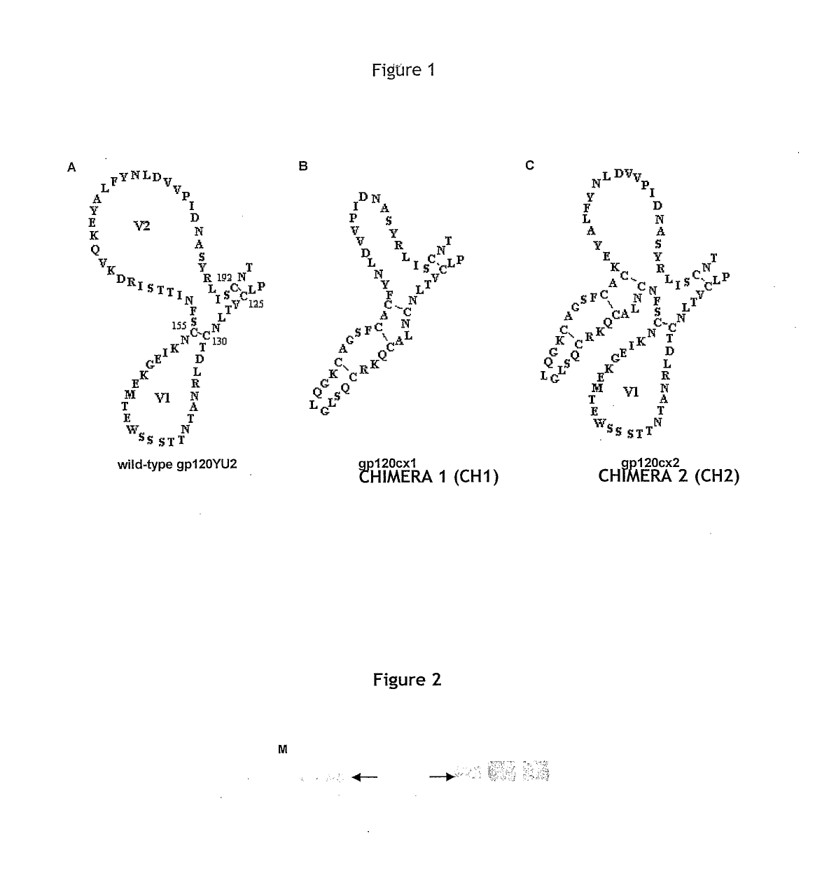

Other characteristics and advantages of the invention will be given in the following examples with reference to FIGS. 1 to 19, which represent, respectively:

FIG. 1: the localization of a CD4 fragment introduced into the V1N2 variable regions of the HIV-1 gp120 (A: wild-type gp120YU2; B: gp120 cx1 (chimera 1 or CH_{S}); C: gp120 cx2 (chimera 2 or CH₂);

FIG. 2: the detection of gp120ΔV1/V2sdCD4 and gp120ΔV2sdCD4 proteins;

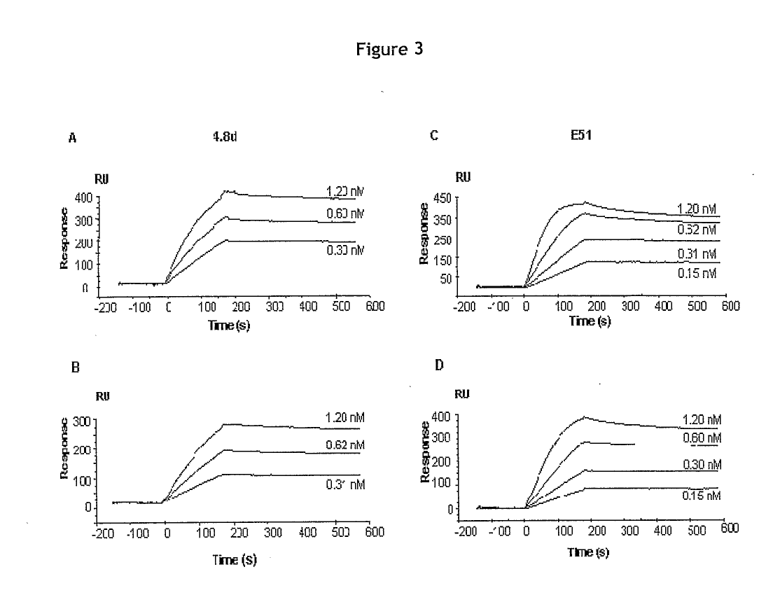

FIG. 3: the sensorgram overlays showing the binding of chimeric proteins to 4.8D and E51 CD4i MAbs by using surface plasmon resonance;

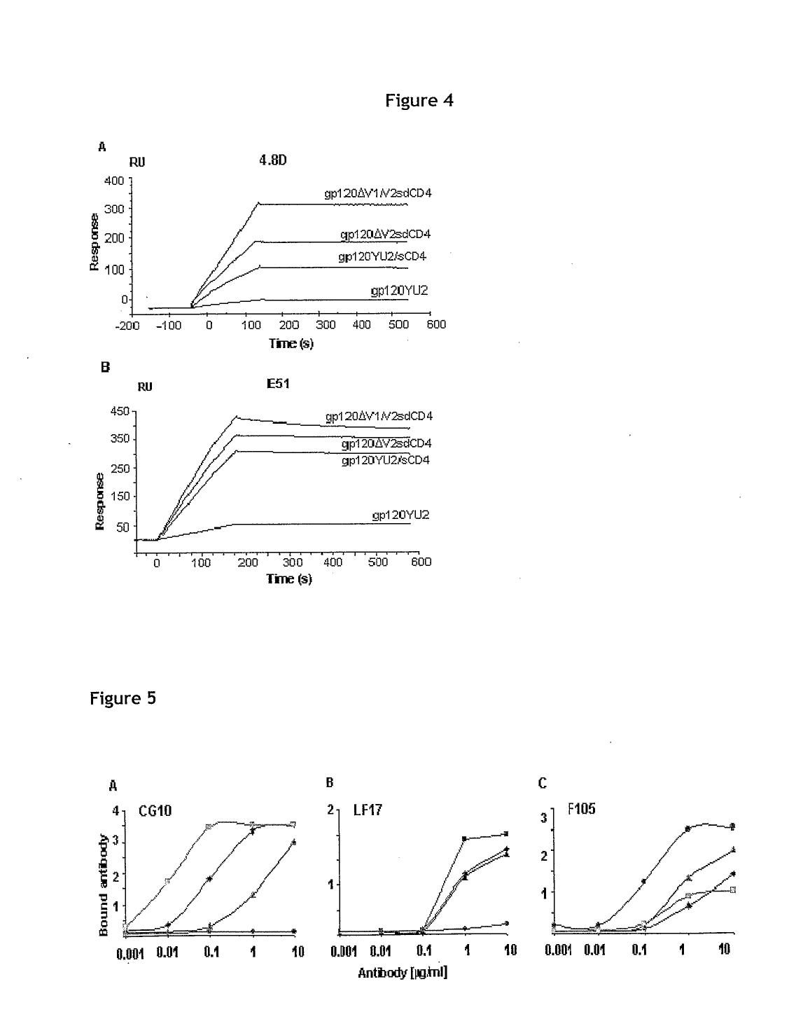

FIG. 4: the sensogram overlays to compare the binding of gp120YU2 with or without sCD4 and chimeric proteins to CD4i MAbs;

FIG. 5: the immunochemical characterization of chimeric proteins by using CG10, LF17 and F105 MAbs;

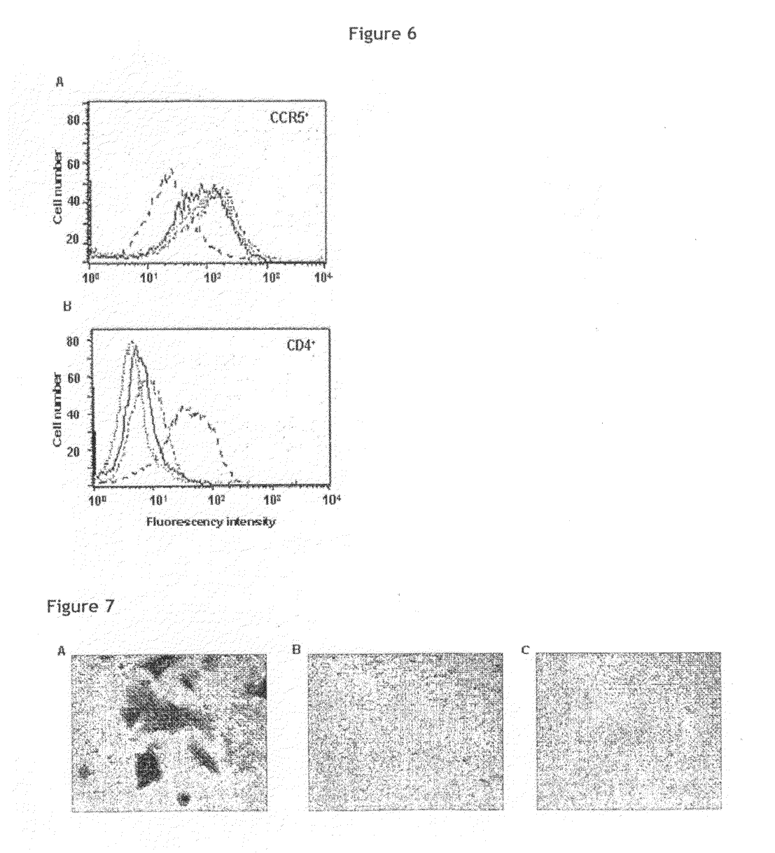

FIG. 6: the binding of gp120 envelope proteins to the CCR5 receptor;

FIG. 7: the assessing of the syncytia formation capacity; and

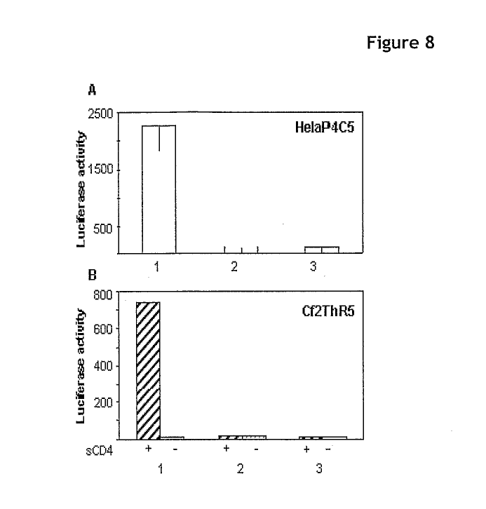

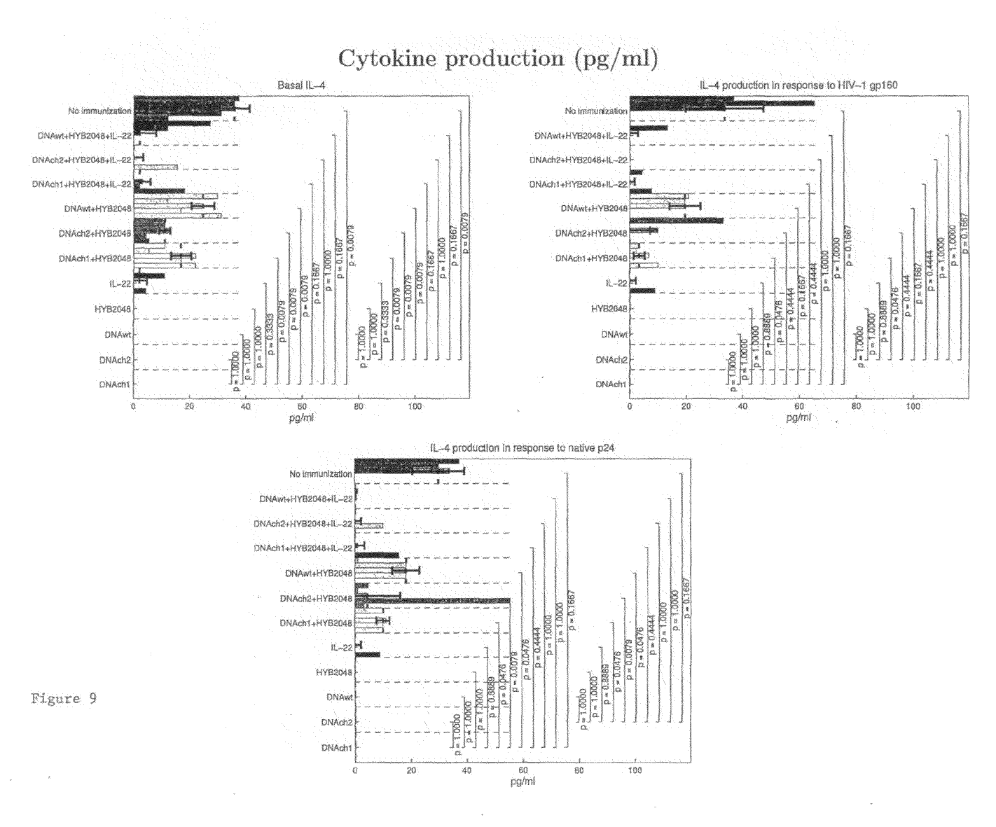

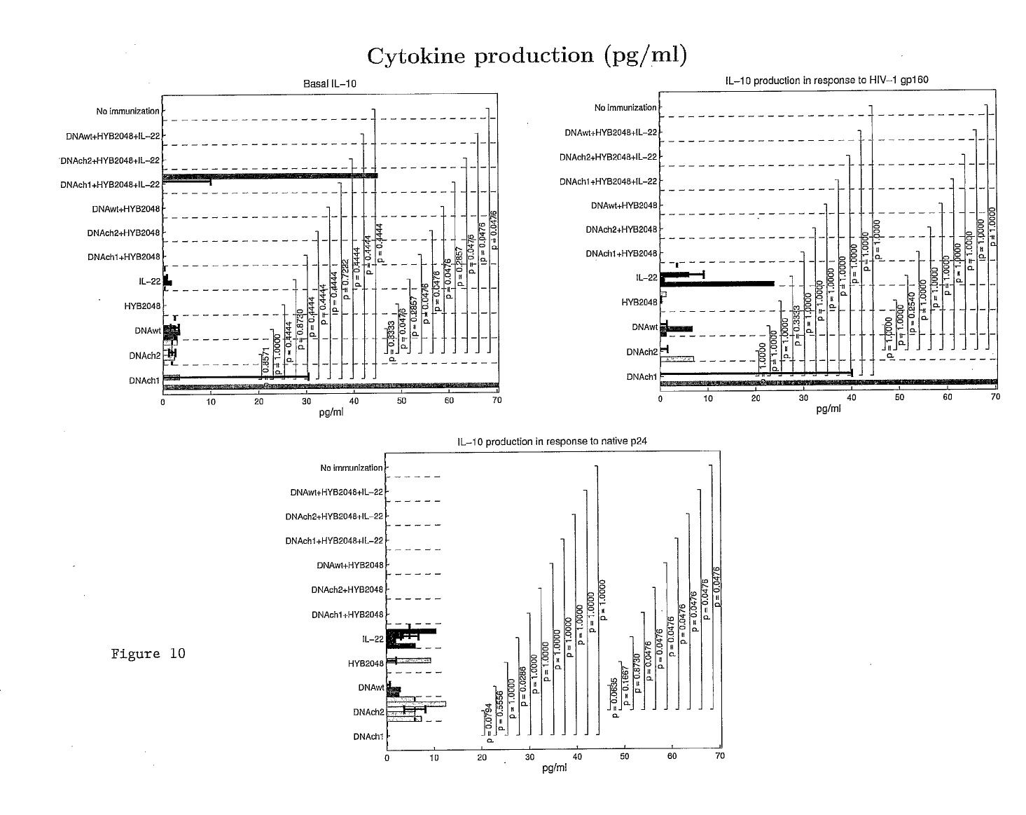

FIG. 8: single round infection assay results;

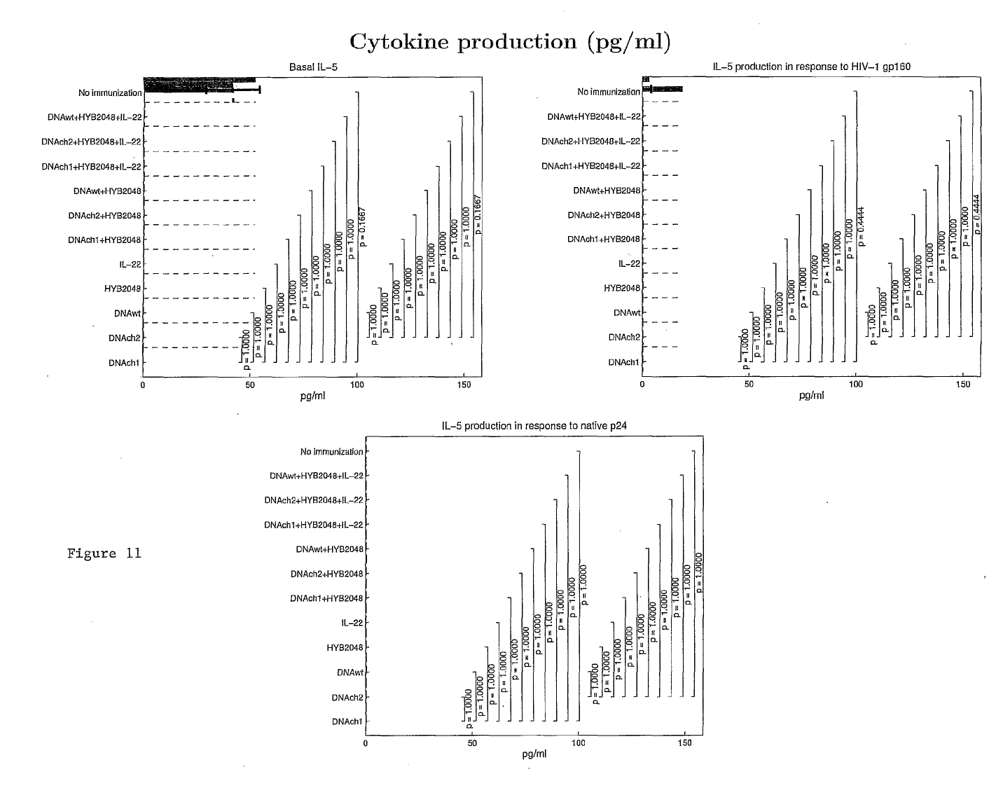

FIG. 9: Interleukin-4 production by spleen cells of female C57/BL6 mice;

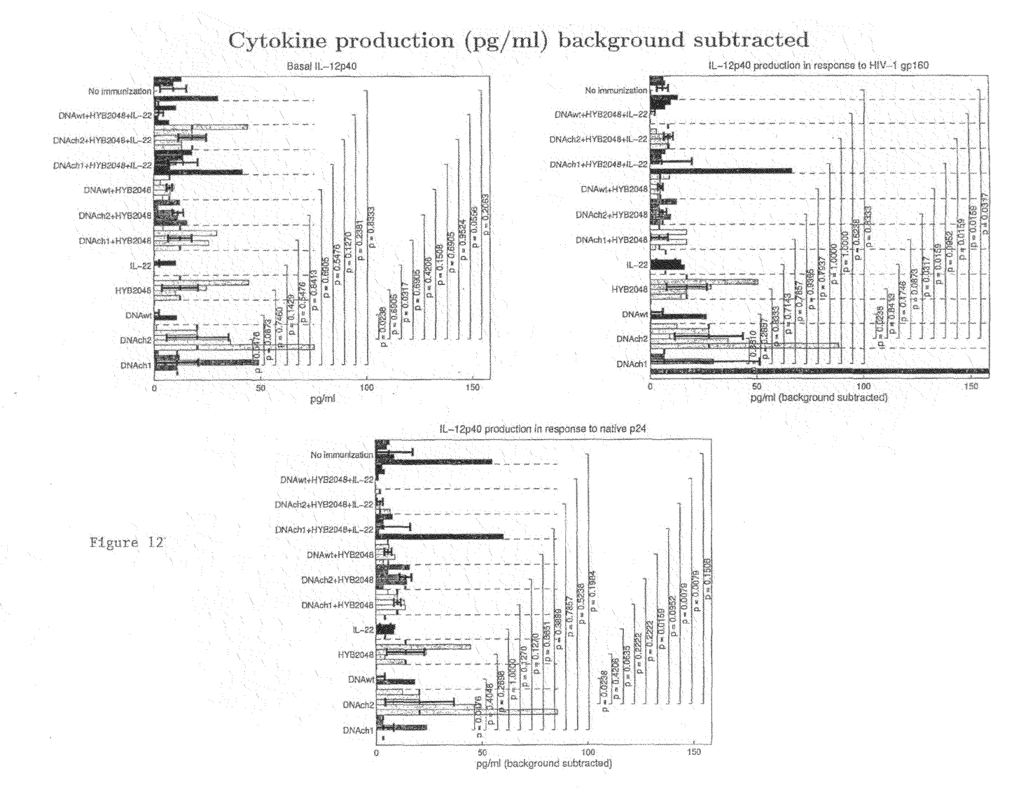

FIG. 10: Interleukin-10 production by spleen cells of female C57/BL6 mice;

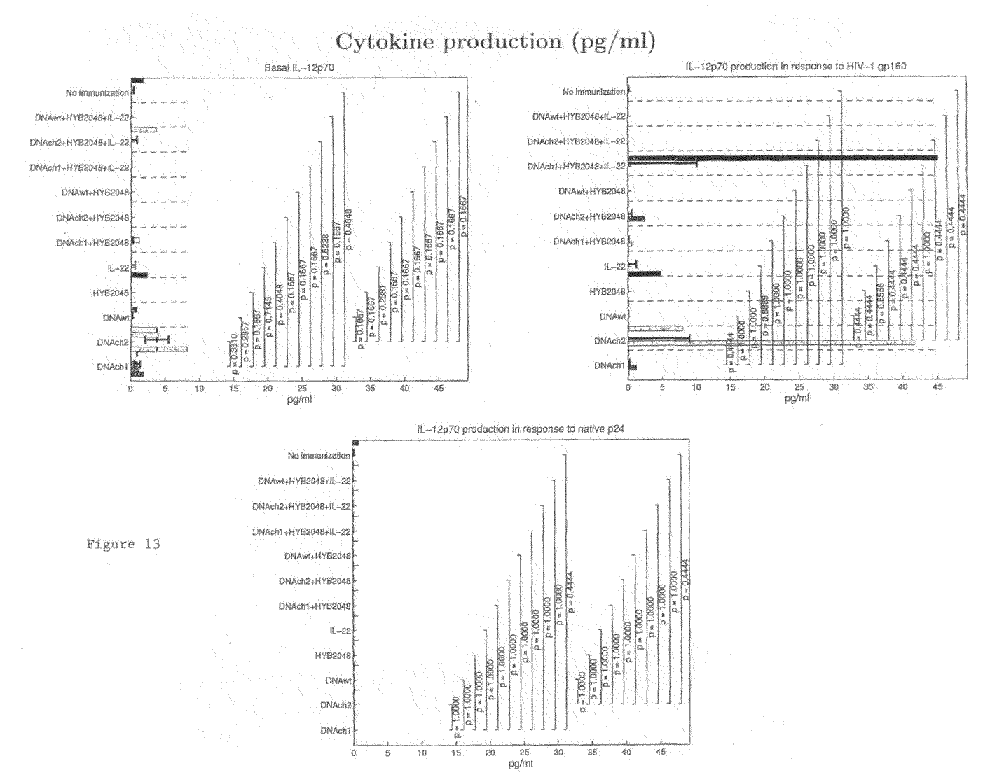

FIG. 11: Interleukin-5 production by spleen cells of female C57/BL6 mice;

FIG. 12: Interleukin-12 p40 subunit production by spleen cells of female C57/BL6 mice;

FIG. 13: Interleukin-12 p70 subunit production by spleen cells of female C57/BL6 mice;

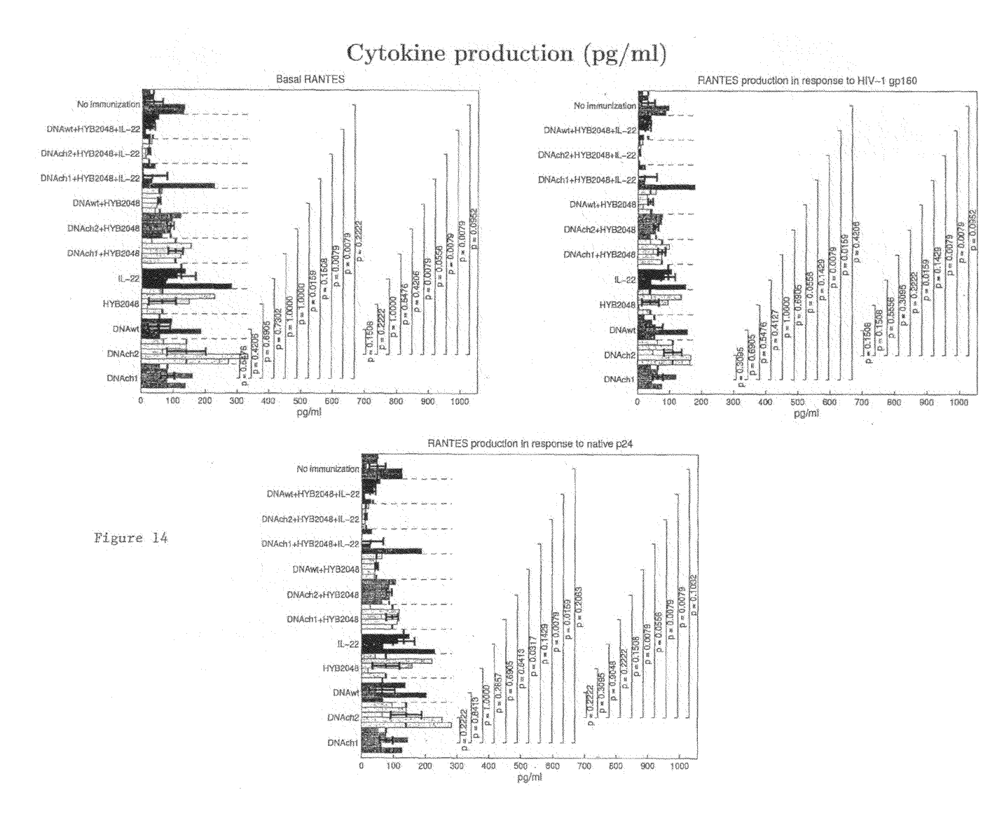

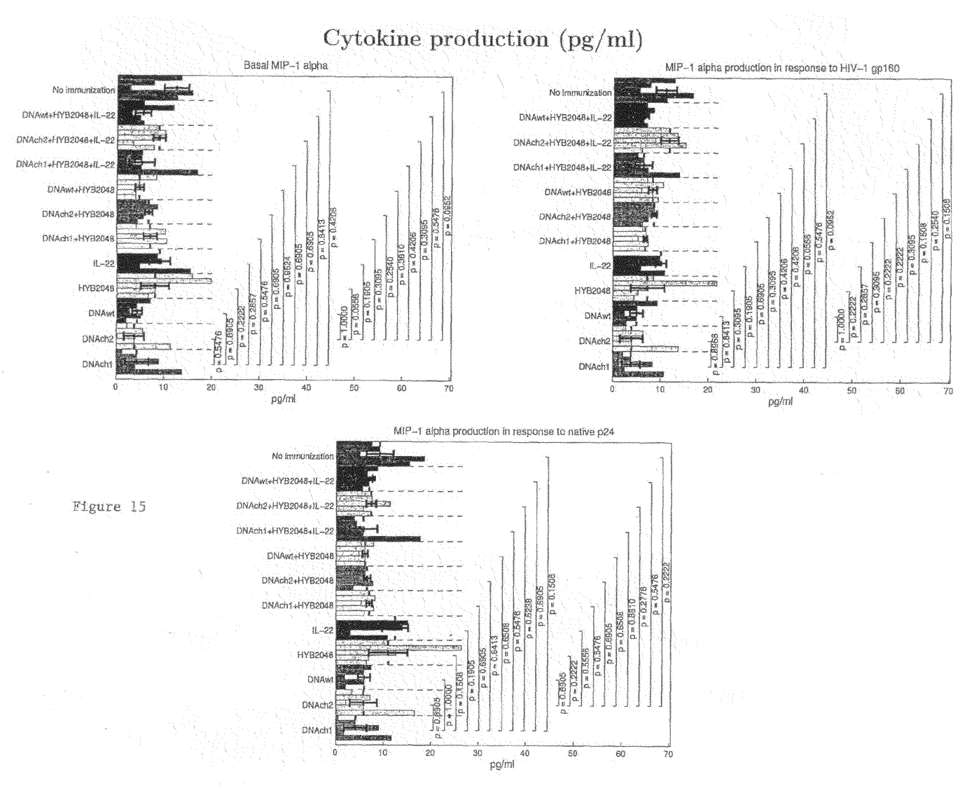

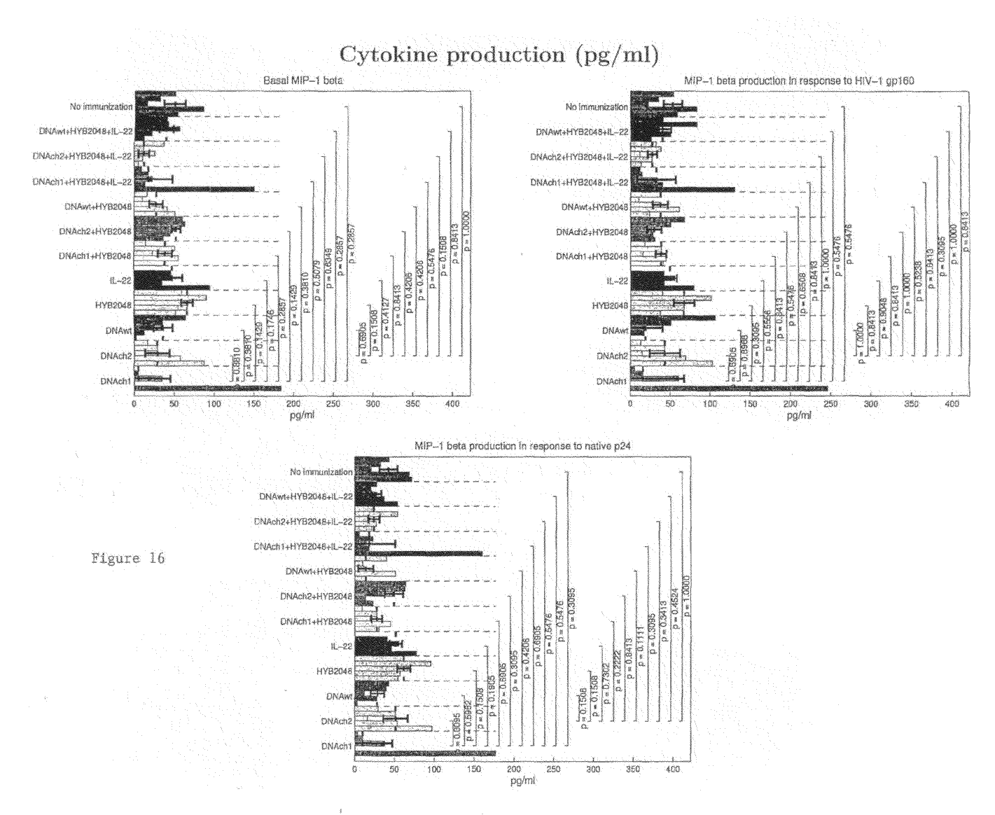

FIG. 14: Rantes production by spleen cells of female C57/BL6 mice;

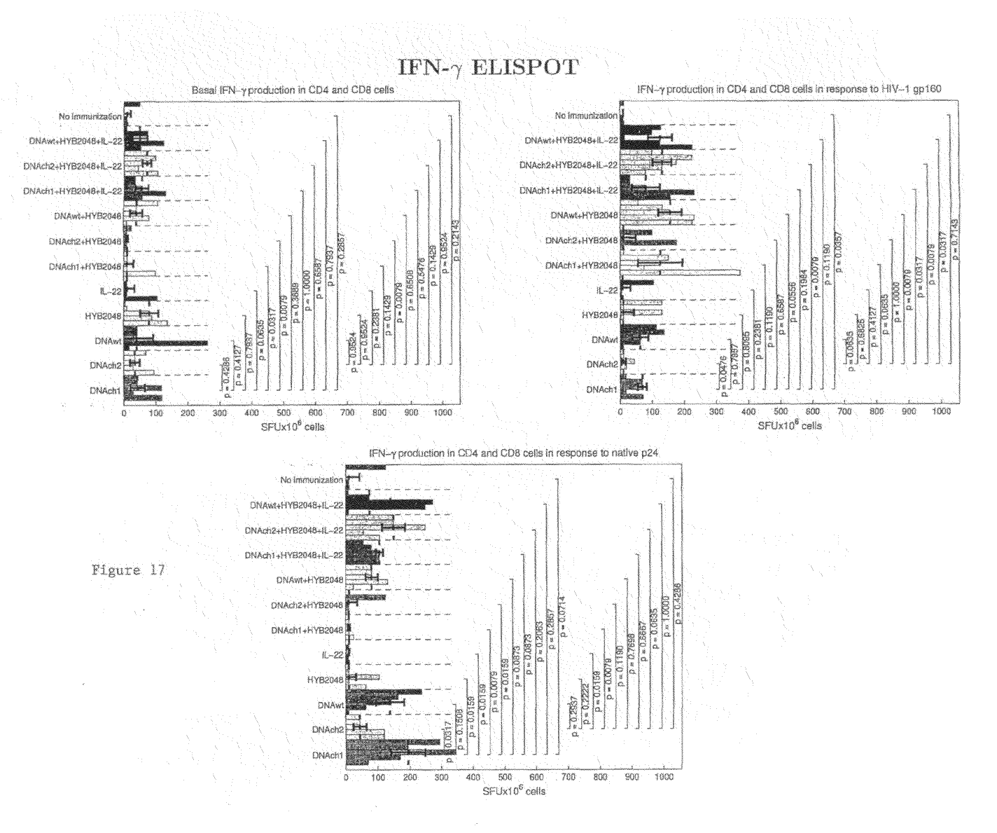

FIG. 15: MIP1-alpha production by spleen cells of female C57/BL6 mice;

FIG. 16: MIP1-beta production by spleen cells of female C57/BL6 mice;

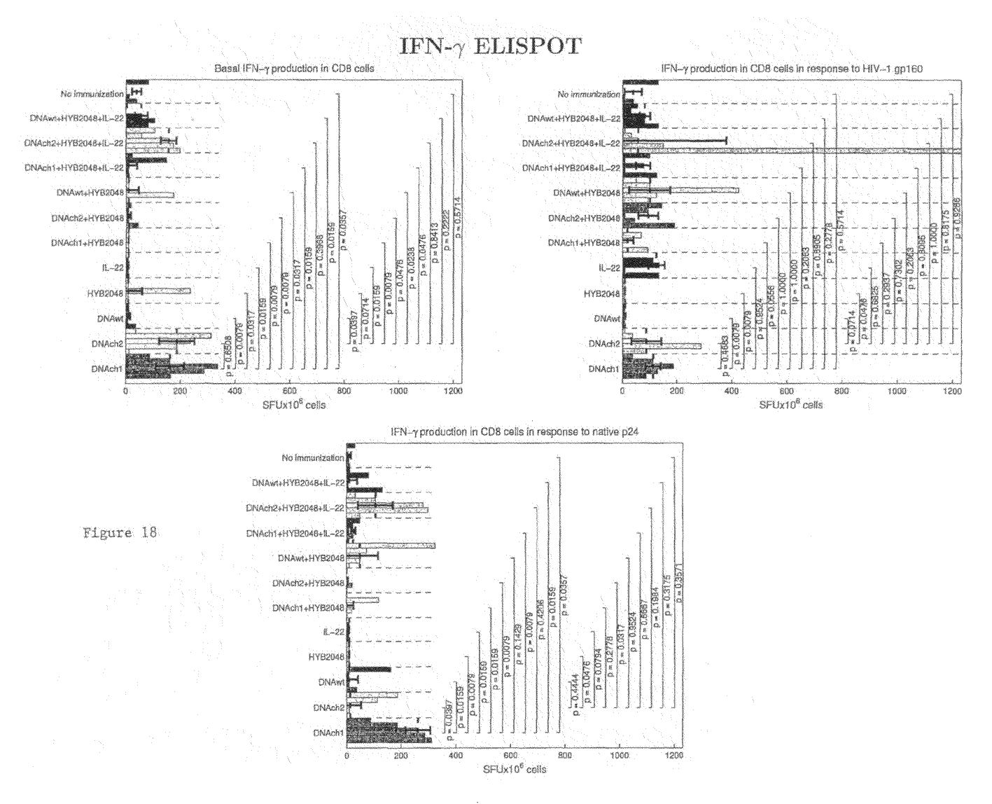

FIG. 17: Interferon gamma production (CD4 and CD8) by spleen cells of female C57/BL6 mice;

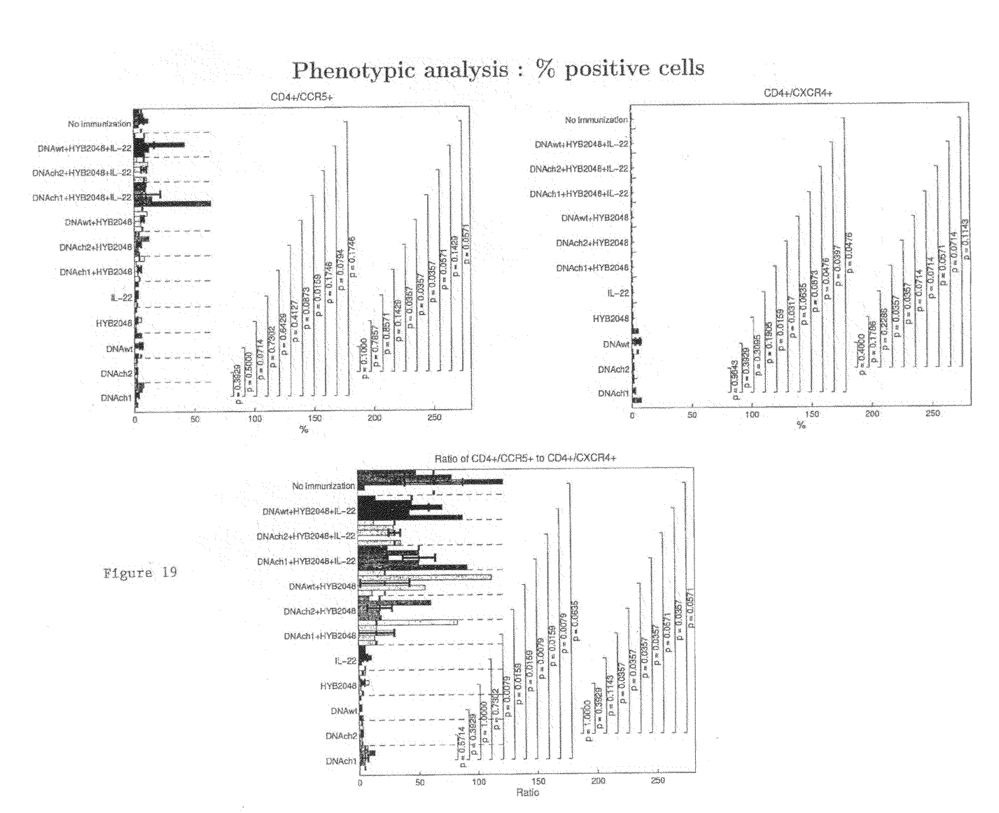

FIG. 18: Interferon gamma production (CD8) by spleen cells of female C57/BL6 mice;

FIG. 19: percentage of co-receptors-expressing CD4+T cells by spleen cells of female C57/BL6 mice;

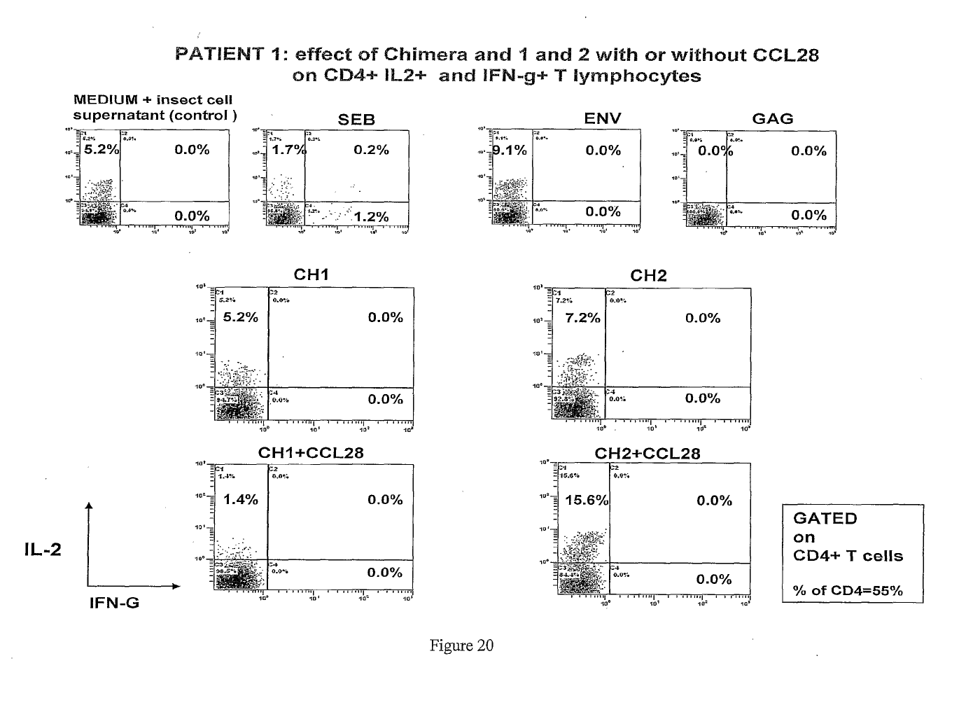

FIG. 20: Effects of Chimera 1 or Chimera 2 with or without CCL28 on the intracellular expression of Interferon-gamma and Interleukin-2 from CD4 T cells.

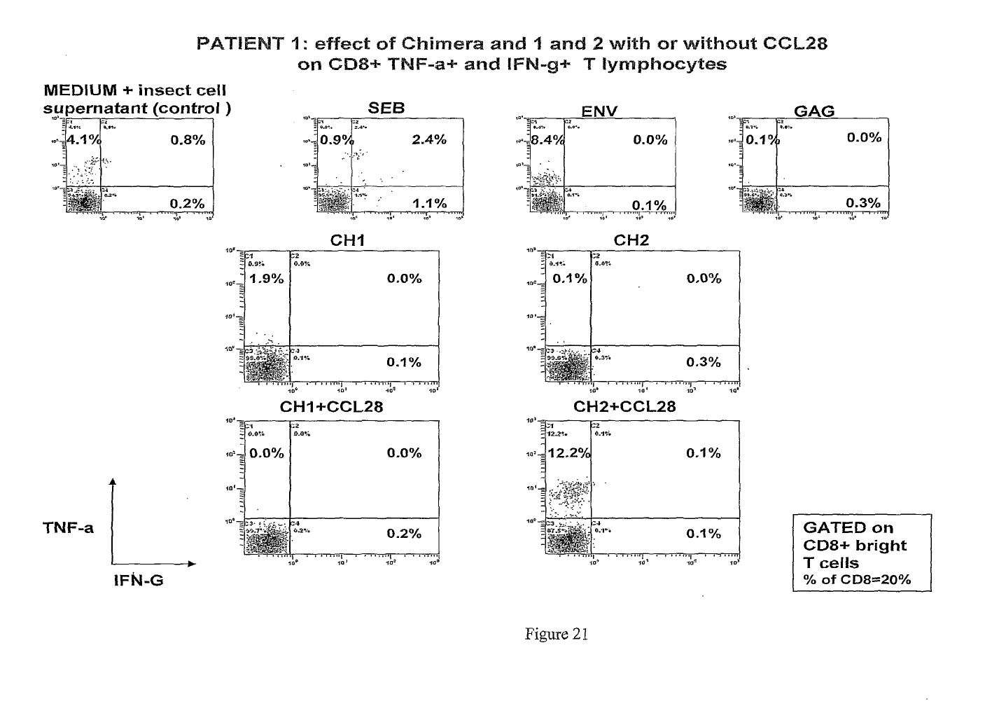

FIG. 21: Effects of Chimera 1 or Chimera 2 with or without CCL28 on the intracellular expression of Interferon-gamma and TNF-alfa from CD8 T cells

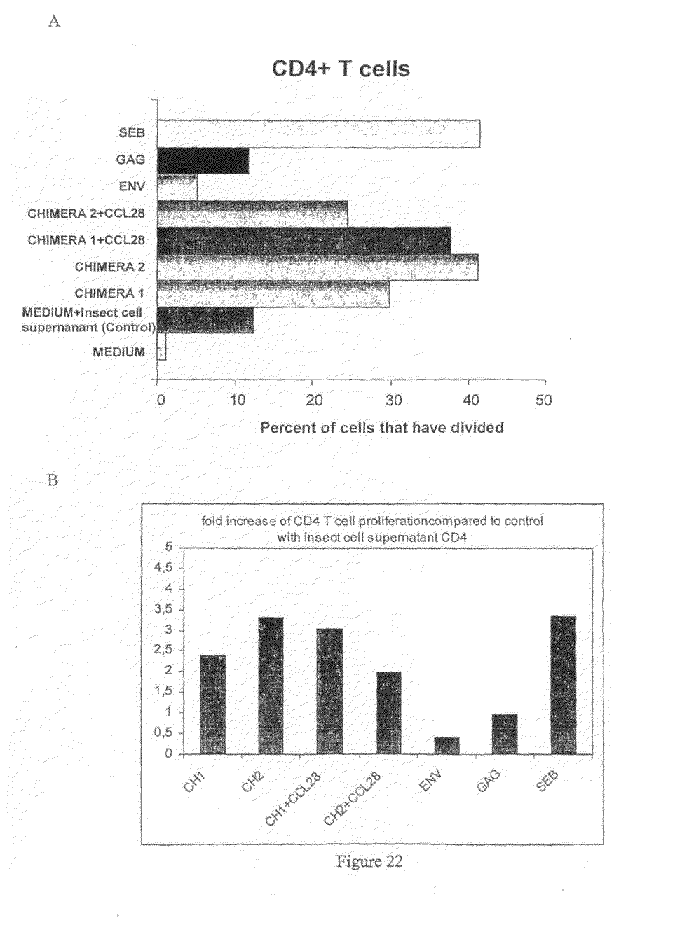

FIG. 22: Effects of Chimera 1 or Chimera 2 with or without CCL28 on the proliferation of CD4+T cells in vitro. A) Percents of CD4+ cells that have divided (CFSE low). B) Fold of proliferation increase compared to the control (insect cell supernatant).

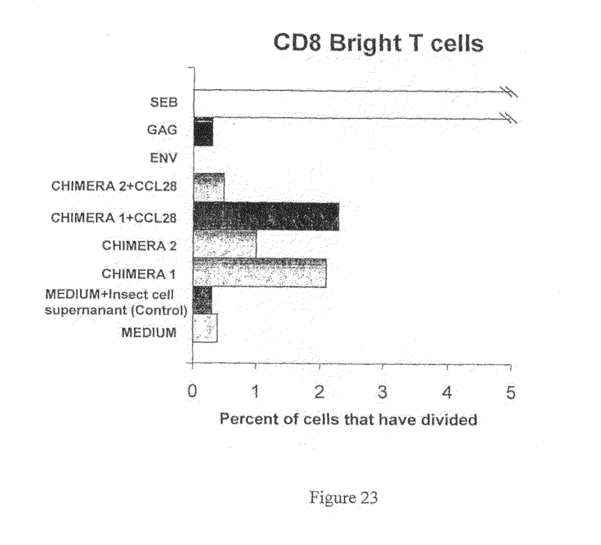

FIG. 23: Effects of Chimera 1 or Chimera 2 with or without CCL28 on the proliferation of CD8 bright T cells in vitro.

### MATERIALS AND METHODS

### Cell Lines and Antibodies

All mamalian cell lines were maintained at 37° C. in a 5% CO₂ atmosphere.

HeLa P4C5 (CD4⁺/CCR5⁺) carrying an integrated HIV LTR-lacZ (1) and Tat-expressing HeLa cell lines were used (P. Charneau and O, Schwartz (Pasteur Institute, Paris, France). These cell lines were grown in Dulbecco modified Eagle medium (DMEM) containing 10% fetal bovine serum (FBS), 2 mM L-glutamine and 100 μg/ml of gentamicin. Both cells were also suplemented with 400 μg/ml of geneticine (G418) plus 50 μg/ml of Hygromycin B and 2 mM Methotrexate respectively.

The human embryonic kidney cells (293T-HEK) were cultured in Improved modified Eagle medium containing 10% FBS, gentamicin and 400 μg/ml G418.

The CD4-positive human lymphoid cells (CEM) obtained from the American type culture collection (Rockville, Md.) were grown in RPM 1640 medium supplemented with 10% FBS and gentamicin.

Canine fetal thymus cells (Cf2ThR5-CCR5⁺) were used (J. Sodroski, Dana Farber Cancer Institute, Boston, Mass.) and were cultured in DMEM containing 10% FBS, gentamicin and 400 μg/ml G418.

Spodoptera frugiperda (Sf9) insect cells were propagated at 28° C. in TC100 medium (GIBCO BRL LIFE Technologies, Gaithersburg, Md.) modified and supplemented with 10% FBS.

Sheep polyclonal anti-gp120 antibody D7324 was obtained from Aalto BioReagents (Dublin, Ireland).

Rabbit anti-gp120 antiserum was made in the laboratory after immunization of a rabbit with a recombinant HIV-1111B gp120 purchased at Intracell Corp (Cambridge, Mass.).

Human CD4i monoclonal antibodies (MAb) 4.8D (2) E51 (3) and LF17 were used (J. Robinson (Tulane University, New Orleans, La.)).

CD4i-specific MAb CG10 was a gift from Dr J. Gershoni, (George Wise Faculty of Life Sciences, Tel Aviv, Israel).

Human MAb F105 (4) was used (M. Posner (New England Deaconess Hospital, Boston, Mass.)).

Soluble CD4 (sCD4) was purchased from Progenies Corp. (Tarrytown, N.Y.).

### DNA Constructs.

HIV-1 YU2 gp160 envelope DNA was amplified by PCR using pTZ-YU2, a plasmid containing the entire sequence of HIV-1 YU2, as template. The following primers having SEQ ID NO 1 to 4, respectively, were used:

SEQ ID NO 1: 5′ CGGGGTACCCCGATGAGAGCGACGGAGATC (containing the underlined KpnI site) as forward; and SEQ ID NO 2: 5′ CGCGGATCCGCGTTATAGCA AAGCTCTTTCCAAGCCC (containing the underlined BamHI site) as reverse for the pSVIIIenv constructions; and SEQ ID NO 3: the 5′CCGCTCGAGCGGATGAGAGCGACGGAGATC (containing the underlined XhoI site) as forward; and SEQ ID NO 4: 5′CCCAAGCTTGGGTTATAGCAAAGCTCTTTCCAAGCCC (containing the underlined HindIII II site) as reverse for the pCEL/E160 constructions.

The PCR products generated using Accuprime Pfx SuperMix (Invitrogen, Carlsbad, Calif.) were inserted into the pGEM-T easy vector (Invitrogen).

DNA fragments encoding gp160 chimeras were constructed by replacing a Nsil-Stul cassette in wild-type gp160 with a fragment generated synthetically corresponding to CD4-structure derived (SEQ ID NO 5 CNLEACQKRCQSLGLQGKCAGSFCAC).

These constructs subsequently were subcloned into pSVIIIenv, replacing the DNA fragment encoding the wild-type gp160 using BamHI/KpnI restriction enzymes, and also into the pCEL/E160 HIV-1 envelope expression vector carrying the cytomegalovirus CMV promoter through the formation of blunt ended by T4 DNA polymerase (New England Biolabs, Beverly, Mass.).

The p119L baculovirus transfer vector for expression of wild type gp120 protein was constructed as previously described (4).

The DNA fragments encoding the gp120 chimeras were constructed using the following primers having sequences SEQ ID NO 6 and 7:

SEQ ID NO 6: 5′ GCGGATCCGCCACCATGACCATCTTATG (containing the underlined HpaI site) as forward; and

SEQ ID NO 7: 5′ CCCAAGCTTGGGTTATCTTTTTTCTCTCTGCA CCA (containing the underlined HindIII site) as reverse. The PCR products were cloned into the p119L exchanging the wild-type gp120 sequence using BamHI/HindIII restriction enzymes.

### Protein Expression and Purification.

The gp120 envelope chimeras were produced by cotransfection of Sf9 insect cells with viral AcSLP10 DNA, expressing the polyhedrin gene under the control of p10 promoter (5) and the p119L transfer vector containing the gp120 chimeras using DOTAP liposomal transfection reagent (Boehringer, Mannheim, Germany). Four days after cotransfection, the supernatants were harvested and recombinant virus plaques were selected by plaque assay (6). Potential recombinant plaques were screened by digestion with HindIII restriction enzyme and Western blot analysis. The envelope proteins were purified from the pooled supernatants using two-type of columns, a Sepharose CoA gel column (Amersham Pharmacia Biotech, Ltd., Buckinghamshire, United Kingdom) and a Dextran Sulphate gel column (Sigma-Aldrich, St. Louis, Mo.) as described previously (7).

### Enzyme-Linked Immunosorbent Assay (ELISA).

Microtiter plates were coated overnight at 4° C. with 10 μg/ml of anti-gp120D7324. Plates were blocked with 3% Bovine serum albumin (BSA) in phosphate-buffered saline (PBS) for 1 h at 37° C. and then washed three times with PBS-0.1% Tween 20 (PBST). Soluble gp120YU2 and chimeric proteins, diluted in PBS-10% FBS, were incubated 1 h at 37° C. in the presence or absence of sCD4 (20 μg/mL) and then were incubated for 1 h at 37° C. with the coated antibody. After, serial dilutions of MAb were incubated with the captured proteins, and bound antibodies were detected with horseradish peroxidase-labeled secondary antibody at 1/10,000 dilution (Amersham Pharmacia Biotech) and o-phenylenediaminesubstrate (Sigma-Aldrich). The colour reaction was stopped after 5-10 min by the addition of 2 M H₂SO₄ and the absorbance at 490 nm was measured.

### Surface Plasmon Resonance Analysis.

The experiments were performed at 25° C. in HBS-EP (HEPES-buffered saline, 3 mM EDTA, 150 mM NaCl, 0.005% nonionic surfactant P20, pH 7.4) using BIACORE 2000 instrument (BIACORE AB, Uppsala, Sweden). To determine the affinity binding of chimeric proteins to CD4i MAb, they were immobilized on a CM4 sensor chip surface using amine coupling. The wild type gp120YU2 were premixed with sCD4 at 37° C. for 1 h before injection. The dissociation phase was followed by a regeneration step with 10 mM HCl. All the sensograms presented were corrected by subtracting the signal from control reference surface. The association and dissociation data were fitted using a 1:1 langmur model.

### Cell Surface Receptor Binding.

A total of 2×10⁵ CEM (CD4⁺/CCR5⁻) or Cf2ThR5 (CCR5) cells were washed twice with PBS-0.3% BSA and incubated with 1 μg/ml of wild type gp120 or chimeric proteins with or without sCD4 (20 μg/ml). The cells were incubated with an anti-gp120 for 1 h at 37° C. Subsequently, after being washed, cells were incubated with a secondary PE-conjugated antibody (Rockland, Gilbertsville, Pa.) at a 1:50 dilution for 45 min at room temperature. The cells were then washed at the same condition and analyzed with a FACSort apparatus (Becton Dickinson, Mountain View, Calif.) using CellQuest software.

### Single-Cycle Infectivity Assay.

pSVIIIenv plasmid expressing the wild-type gp160 or chimeric proteins were transfected with the pCMV Gag-PoI packaging plasmid and the pHIV-luc vector into 293T-HEK cells using Effectene transfection reagent (Qiagen Inc. Valencia, Calif.). Thirty hours after transfection, the cell supernatants were collected, filtered through 0.45-μm-pore-size filters and concentrated by ultracentrifugation. For infection of Cf2ThR5 and HeLa P4C5 cells, standardized amounts of pseudotyped virus (200 ng of p24 protein) were incubated with the cells at 37° C. for 48 h in 96-well luminometer plates (Dynex Technologies, Chantilly, Va.). Luciferase activity was determined by adding 100 μl luciferase assay buffer and 50 μl luciferase substrate (Promega, Madison, Wis.) to the cell lysate and measuring the intensity of chemiluminescence in the TECAN Genios luminometer.

### Cell-Cell Fusion Assay.

HeLa-tat grown to 70% confluence in 6-well plates were transfected with 1 μg of an envelope protein-expressing plasmid using Lipofectamine reagent (Invitrogen) as recommended by the manufacturer. An equivalent amount of HeLa CD4⁺/CCR5⁺LTR-lacZ cells were added at 48 h post-transfection. After overnight coculture, adherent cells were fixed with 0.5% glutaraldehyde for 10 min and stained for β-galactosidase activity with the X-Gal (5-bromo-4-chloro-3-indolyl-β-D-galactopyranoside) substrate overnight at 37° C. The total number of blue-stained foci per well were counted and photomicrographs were obtained.

### Results

Construction and Purification of gp120ΔV1/V2sdCD4 and gp120ΔV2sdCD4 Proteins.

The chimeric protein of the invention is a single chain glycoprotein exhibiting a CD4i conformation with a sdCD4 inserted into the HIV-1 gp120YU2 sequence. The CD4-derived peptide has 28 amino acids and mimics the CDR2-like loop of human CD4 receptor which is the major binding site for the gp120 envelope glycoprotein. The DNA sequence of this CD4-derived peptide, generated synthetically from oligonucleotides, was cloned into the V1N2 variable regions of gp120YU2 glycoprotein as shown in FIG. 1: (A) Predicted amino acid sequence of the V1/V2 loops of wild type gp120YU2. (B) The gp120ΔV1/V2sdCD4 envelope protein wherein the V1 and part of V2 loop (16 amino acids) was replaced with sdCD4 sequence. C) In gp120ΔV2sdCD4 protein, the sdCD4 was inserted in V2 loop by replacing the region encoding 156-168 amino acids. The entire V1 loop was retained. The amino acid numbers correspond to the cysteine residues.

gp120ΔV1/V2sdCD4 chimeric protein was constructed by replacing the V1 loop and 16 amino acid residues from V2 loop with the sdCD4 sequence.

The gp120ΔV2sdCD4 chimeric protein retains the entire V1 loop, but sdCD4 replaces the region encoding 156-168 amino acids in V2 loop.

Wild type and chimeric gp120 sequences were cloned into the baculovirus transfer vector, p119. To facilitate the efficient secretion of recombinant proteins, the native HIV signal sequence was replaced by a new signal sequence isolated from the ecdysteroidglycosyltransferase (EGT) gene of the Autographa californica baculovirus (7). All constructed chimeras expressed approximately the same amount of gp120 envelope proteins as determined by ELISA and Western blot analysis. It appears that inserted sdCD4 sequence did not affect the level of expression. Furthermore, the proteins were purified from Sf9 insect cell culture supernatants using two-step purification procedure and then characterized by SDS-polyacrylamide gel electrophoresis. The results are given on FIG. 2. The gel was either stained with Coomassie blue (FIG. 2A) or processed for Western blot analysis (FIG. 2B) using anti-gp120 D7324 antibody (arrows at the right indicate the position of the envelope proteins).

Chimera corresponding bands exhibit an apparent molecular weight (MW) of 100 kDa is consistent with those of the wild-type gp120 envelope, expressed under the same conditions.

### Antigenic Properties of Chimeric Proteins.

Structural integrity of the chimeric proteins was determined by enzyme-linked immunosorbent assay (ELISA) with a panel of MAbs against known epitope specificities (D7324, CG10, LF17 and F105). The ELISA was performed as described in Materials and Methods with the CG10 and LF17 MAbs, directed against the CD4-inducible binding site.

The results are given on FIG. 5. The wild type gp120 (circle), gp120/sCD4 (square), gp120ΔV1/V2sdCD4 (rhombus) and gp120ΔV2sdCD4 (triangle, 4) proteins were captured by the D7324 antibody and the MAbs CG10 (A), LF17 (B) and F105 (C) were added at the indicated concentration. The amount of bound antibodies were detected by specific F(ab′)₂-HRP conjugate and represented as optical density at 490 nm.

These antibodies recognize only the gp120/sCD4 complex, but do not recognize gp120 in the absence of sCD4. Both chimeric proteins were recognized by

CG10, however, gp120ΔV1/V2sdCD4 chimeric protein was recognized more efficiently than gp120ΔV2sdCD4 (FIG. 5A), suggesting that this epitopes was less exposed. The LF17 bound both chimeric proteins with similar efficiency, but less compared to the wild type gp120/sCD4 complex as shown in FIG. 5B.

Since the formation of gp120/sCD4 complex blocks the exposure of CD4 binding site on gp120, the ability of a CD4-binding site antibody to bind chimeric proteins was examined.

The human F105 MAb better bound to wild type gp120 protein than to gp120/sCD4 complex and chimeric proteins as shown in FIG. 5C. The gp120ΔV2sdCD4 was recognized more efficiently than the complex and gp120ΔV1/V2sdCD4 protein.

These data were consistent with previous results, in which gp120ΔV1/V2sdCD4 protein exposed better to CD4i epitope and therefore less the CD4-binding epitopes.

Said results demonstrate that the inserted sdCD4 causes conformational changes in chimeric proteins leading to the exposure of CD4i epitopes, especially in gp120ΔV1/V2sdCD4 protein.

Interestingly, anti-CD4, ST4 and BF5 MAbs (able to specifically recognize on CD4 molecule the epitope that is involved in the binding to gp120 and a neighbourhood epitope respectively) does not recognize any of the two gp120/CD4 chimeras. This means that these molecules have small probabilities to induce anti-CD4 antibodies after immunisation.

Moreover anti-gp120 MAbs respectively directed against V2 loop, the conformational epitope 2G12, C- and N-terminal peptides were able to recognize these epitope on chimeras similarly as they recognize it on wild type gp120.

### BIACORE Analyses of the Binding Strength of the Chimeric Proteins to Immobilized CD4i MAb.

The quantitative binding of chimeric proteins to CD4i MAb was performed by determining the association (kₐ) and dissociation rates (k_{d}) and the equilibrium dissociation constant (KD) by surface plasmon resonance (SPR). Various concentrations of either wild type gp120YU2 with or without sCD4, gp120ΔV1/V2sdCD4 or gp120ΔV2sdCD4 proteins were passed over immobilized 4.8D and E51 CD4i MAbs.

The results are summarized in the following Table:

TABLE 1
Kinetic parameters of molecular interaction between chimeric gp120
and anti-CD4i MAbs. Association (ka) and dissociation rate constant (kd) and the
equilibrium dissociation constant (KD). Rmax represents the maximum protein
binding capacity to the immobilized antibody.

| | | | | |
|---|---|---|---|---|
| kₐ (M⁻¹s⁻¹) | k_{d} (s⁻¹) | KD (nM) | Rmax | Chi² |

E 4.8

| | | | | | |
|---|---|---|---|---|---|
| gp120/sCD4 | 1.77 ± 0.05 × 10⁵ | 2.12 ± 0.02 × 10⁻⁴ | 1.2 ± 0.005 | 603 | 0.427 |
| gp120ΔV1/V2sdCD4 | 9.45 ± 0.02 × 10⁶ | 1.49 ± 0.04 × 10⁻⁴ | 0.015 ± 0.0004 | 415 | 22.8 |
| gp160ΔV2M9 | 5.2 ± 0.01 × 10⁶ | 1.97 ± 0.03 × 10⁻⁴ | 0.038 ± 0.0007 | 382 | 8.01 |

E51

| | | | | | |
|---|---|---|---|---|---|
| gp120/sCD4 | 7.96 ± 0.12 × 10⁵ | 1.69 ± 0.03 × 10⁻⁴ | 0.21 ± 0.005 | 788 | 14.2 |
| gp120ΔV1/V2sdCD4 | 4.27 ± 0.04 × 10⁷ | 5.16 ± 0.07 × 10⁻⁴ | 0.012 ± 0.0002 | 410 | 31.8 |
| gp160ΔV2M9 | 3.08 ± 0.02 × 10⁷ | 3.98 ± 0.04 × 10⁻⁴ | 0.013 ± 0.0001 | 384 | 8.5 |

The resultant sensorgrams are shown in FIG. 3A-D. Different concentrations of gp120V1/V2sdCD4 (A, C) and gp120V2sdCD4 (B, D) proteins were passed over immobilized 4.8D (A, B) and E51 (C, D) at a flow rate of 50 μl/min. MAbs were immobilized at 2000 and 1100 RU to a CM4 sensor chip respectively. RU, response units. The summary of the binding constants are presented in FIG. 3E.

The calculated KD (kd/ka) for gp120ΔV1/V2sdCD4 and gp120ΔV2sdCD4 were 0.015±0.0004 nM and 0.038±0.0007 nM respectively for 4.8d MAb, while the corresponding KD for gp120/sCD4 complex was 100-fold higher.

This difference results primarily from a higher kₐ for these chimeric proteins.

Similar results were observed with another CD4i MAb, E51. The KD values for gp120ΔV1/V2sdCD4 and gp120ΔV2sdCD4 were 0.012±0.0002 nM and 0.013±0.0001 nM respectively, suggesting a high affinity binding for E51 MAb.

FIG. 4 show sensogram overlays for interactions between the gp120 with or without sCD4 and chimeric proteins to CD4i MAb. The 4.8d (A) and E51 (B) MAbs were immobilized at 2000 and 1100 RU proteins were passed at a flow rate of 30 μl/min. The gp120YU2 protein was incubated with a threefold molar excess of sCD4 for 1 h at 37° C. before injection.

Wild type gp120YU2 in the absence of sCD4 showed no significantly binding for 4.8d but bound slightly to the E51 MAb. As expected, sCD4 increased binding for both CD4i antibodies with high affinity. Both chimeric proteins, however, bound to 4.8d and E51 with higher affinity, showing that the inserted sdCD4 do not affect the correct folding of the proteins.

Binding of gp120 Chimeric Proteins to CCR5 and CD4 Receptor.

Since the above results shown by ELISA and BIACORE technology that chimeric proteins, especially gp120ΔV1/V2sdCD4, have a CD4-bound conformation, experiments were carried out assess if these proteins bind to CCR5 chemokine receptor.

Cf2thR5 cells, expressing high amounts of CCR5, were incubated with wild type and chimeric gp120 proteins in the presence or absence of sCD4. The results are given on FIG. 6, which show the binding activity of gp120 to CCR5 receptor (A) and CD4 (B) receptors expressed in Cf2ThR5 and HeLa P4C5 cell lines respectively. The cells were incubated either with gp120 (_{— —}), gp120/sCD4 ( ) gp120ΔV1/V2sdCD4) ( . . . ) or gp120ΔV2sdCD4 ( - - - ) proteins for 1 h at 37° C. The bound proteins were detected using an anti-gp120 antiserum (A) and the CG10 MAb (B), and revealed by using a PE-coupled secondary antibody. Stained cells were washed and analyzed by flow cytometry.

As shown in FIG. 6A, both chimeric proteins bind as well to the CCR5 in the absence of sCD4 as the gp120/sCD4 protein complex, suggesting the correct exposition of co-receptor binding site and the CD4i epitopes. The presence of sCD4 does not increase the binding of gp120ΔV1/V2sdCD4 to CCR5, but it slightly increases the binding of gp120ΔV2sdCD4.

In a second assay, the ability of chimeric proteins to bind CD4 receptor were examined. CEM cells expressing CD4 protein but not CCR5 protein were incubated with wild type and chimeric proteins for 1 h at 37° C. Both recombinant proteins exhibited decreases in CD4 binding compared with the wild type protein (FIG. 6B). The ability of gp120ΔV1/V2sdCD4 to bind CD4 receptor was similar to gp120/sCD4 complex protein, however, gp120V2sdCD4 bound better to CD4.

### Cell-Cell Fusion Assay of Cell-Surface-Expressed HIV-1 Chimeric Envelops.

To determine whether the chimeric proteins maintain the fusion activity leading to syncytium formation, HeLa-Tat cells were transfected with pCEL envelope expression vector. The transfected cells were co-cultured with HeLa P4C5 cells expressing CD4 and CCR5 receptor. HeLa-Tat cells were transfected either with the wild type gp160 (A), gp160ΔV1/V2sdCD4 (B) or gp160ΔV2sdCD4 (C) envelope constructs (FIG. 7). At 48 h post transfection, the cells were cocultured with HeLa P4C5 cells carrying an integrated HIV LTR-/acZ and expressing CCR5 and CD4 receptor. Cells were fixed and stained with X-Gal. Syncytia were counted following an overnight incubation. None of the chimeric proteins yielded detectable formation of syncytia. The wild type under these conditions formed over 89 syncytia.

### Infection by Virus Particles Containing Chimeric Envelope Glycoproteins.

The ability of chimeric proteins to mediate viral entry was analysed using a plasmid expressing either wild-type or chimeric envelope glycoproteins that complement an env-defective HIV-1 provirus encoding luciferase reporter gene. Recombinant virions produced in 293T cells were incubated either with HeLa P4C5 and Cf2ThR5 cells stably expressing CCR5 and CCR5/CD4 respectively, and the luciferase activity in the target cells was measured. The recombinant viruses expressing the firefly luciferase and either the wild type gp160 (1), gp160ΔV1/V2sdCD4 (2) or gp120ΔV2sdCD4 (3) envelope constructs were incubated either with HeLa CD4⁺CCR5⁺ (A) or Cf2Th-CCR5 (B) cells (FIG. 8). Luciferase activity was measured after 48 hours as described in materials and methods. These data represents four independent experiments.

Virus pseudo-particles containing functional wild type envelope protein infected HeLa P4C5 and also Cf2ThR5 cells, but only in the presence of sCD4 as expected. However, chimeric proteins were not able to infect neither Cf2ThR5 nor HeLa P4C5 cells, suggesting that both proteins appear to be defective for some aspects of the membrane fusion and replication process.

Taken together our results, it is possible to consider that this kind of proteins could be considered as good prototype for immunisation because the major epitopes of wild type of gp120 from HIV-1 YU2 are conserved in these constructions.

### In Vivo Immunogenicity of Chimera 1 and Chimera 2 Using Chimeric Plasmids in Mice.

The goal of the experiment was to evaluate the effects on the immune response of chimera expressing small CD4-derived peptides that interfere with the binding of HIV gp120 to its receptors (chimera 1 and chimera 2) To reach this goal, mice were immunized with chimeric HIV-1 envelope glycoproteins containing a CD4-derived peptide into the V1/V2 variable region to avoid linkers or cross-linking agents. Two different adjuvants were used: a CpG-like motif (HYB2048) and interleukin-22.

1.1. Rationale for using CpG-like motif (HYB2048) and IL-22 in combination with Chimera 1 or Chimera 2

A wide range of investigations over the last several years have demonstrated that bacterial DNA and synthetic oligodeoxynucleotides containing unmethylated CpG motifs (CpG DNA) are potent adjuvants that induce innate immune cells to produce Th1 cytokines, promote CTL responses and boost the production of. immunoglobulins by B lymphocytes. These effects of CpG DNAs are likely secondary to their interaction with the Toll-like receptor 9 (TLR9) known to stimulate differentiation and maturation of dendritic cells. Use of CpG DNAs as adjuvants in combination with a variety of vaccines, antigens and immunogens has been evaluated for enhancing both arms of specific immunity. For example, addition of a synthetic CpG DNA to an inactivated gp120-depleted HIV-1 immunogen provides a boost of both cell-mediated and humoral virus-specific immune responses in rodents and in primates. Recently novel synthetic TLR9 agonists have become available. One of such agonists is HYB20482055, referred to as Amplivax™. HYB20482055 is an immunomodulatory oligonucleotide (IMO) consisting of novel 3′-3′-linked structure and synthetic CpR (R=2′-deoxy-7-deazaguanosine) motif. The novel 3′-3′-structure provides greater stability against ubiquitous nucleases because of the absence of free 3′-ends and the presence of two accessible 5′-ends in HYB20482055 provides enhanced TLR9 activation compared with conventional CpG DNAs. Additionally, the synthetic CpR dinucleotide motif has been shown to induce a distinct cytokine induction profile characterized by higher IL-12 and lower IL-6 compared with natural CpG dinucleotide motif.

IL-22 is a member of the human type I IFN family, which includes IL-10. IL-22 has the potential to interact with IL-10 since it binds to the IL-10R2c chain with IL-22R1 in its receptor complex. IL-22 mediates inflammation and binds class II cytokine receptor heterodimers IL-22 RA1/CRF2-4. This cytokine is also involved in immuno-regulatory responses. IL-22 inhibitors have been proposed to treat inflammatory disorders such as arthritis. Recent data suggest that IL-22 is capable of inducing innate immunity these was completed by data showing that IL-22 participates to the protection against HIV in exposed uninfected individuals and thus appears to be a suitable target for vaccine adjuvant.

The immunogenicity of HIV-1 Immunogen, a gp120-depleted whole killed virus vaccine candidate formulated with Incomplete Freund's Adjuvant (HIV-IFA), have been evaluated in a mouse model by itself and when combined with HYB2055 and HYB2048. Compared to HIV-IFA alone, immunization with HIV-IFA and HYB2055 combination elicited strong production of HIV- and p24-specific IFNγ, RANTES, MIP 1α, and MIP 1β, as well as high titers of HIV- and p24-specific antibodies. Inclusion of HYB2055 and HYB2048 also reduced levels of IL-5 produced by HIV-IFA alone. HYB2048 enhances the immunogenicity of HIV-IFA and shifts responses towards a type 1 cytokine profile. The immune enhancing effects of HYB2055 and HYB2048 adjuvant were dose-dependent.

Therefore, CpG-like motif or IL-22 appears to be potential adjuvant candidates to increase the immune response to Chimera 1 and/or Chimera 2.

The aim of the invention is study is to asses whether the ImmunoChimera used alone as plasmid is capable of inducing HIV specific immune response and to examine whether HYB20482048 and/or IL-22 would enhance such responses

1.2. METHODS

1.2.1. Chimeras Constructs

Wild type and chimeric gp120 sequences were cloned into the baculovirus transfer vector, p119L. To facilitate the efficient secretion of recombinant proteins, the native HIV-1 signal sequence was replaced by a new signal sequence isolated from the ecdysteroid glycosyltransferase (EGT) gene of the Autographa californica baculovirus. The gp120 envelope chimeras were produced by cotransfection of Sf9 insect cells with viral AcSLP10 DNA, expressing the polyhedrin gene under the control of p10 promoter and the p119L transfer vector containing the gp120 chimeras using DOTAP liposomal transfection reagent. Four days after cotransfection, the supernatants were harvest and recombinant virus plaques were selected by plaque assay. Potential recombinant plaques were screened by digestion with HindIII restriction enzyme, Western blot analysis and ELISA. The reagents used in the experiments were supernatants of recombinat baculovirus-infected Sf9 cells containing Chimera 1 and Chimera 2.

1.2.2. Protocol Schema

70 female C57/BL6 mice; 6-8 weeks of age (N=5/group) has been immunized IM with immunoChimera 1 or 2 (200 μg per mouse: 2×50 μg per quadriceps) and/or the mouse oligonucleotide HYB20482048 (100 μg per mouse: 50 μg per quadriceps) and/or IL-22 (100 μg per mouse: 50 μg per quadriceps). After primary immunization, mice have been boosted 3 weeks later.

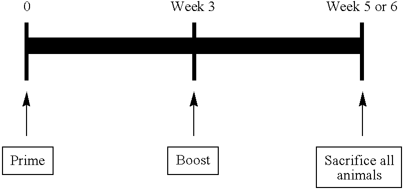

| Prime (time 0) | Boost (3 weeks) |
|---|---|
| DNAch1 | DNAch1 |
| DNAch2 | DNAch2 |
| DNAwt | DNAwt |
| HYB2048 | HYB2048 |
| IL-22 | IL-22 |
| DNAch1 + HYB2048 | DNAch1 + HYB2048 |
| DNAch2 + HYB2048 | DNAch2 + HYB2048 |
| DNAwt + HYB2048 | DNAwt + HYB2048 |
| DNAch1 + HYB2048 + IL22 | DNAch1 + HYB2048 + IL22 |
| DNAch2 + HYB2048 + IL22 | DNAch2 + HYB2048 + IL22 |
| DNAwt + HYB2048 + IL22 | DNAwt + HYB2048 + IL22 |

DNAch1 = pcDNAgp160 chimeric protein 1
DNAch2 = pcDNAgp160 chimeric protein 2
DNAwt = pcDNAgp160YU2

1.2.3. Immunological Analyses

Immunological analyses have been carried out on fresh splenocytes stimulated in vitro for 4 days in medium alone; with native p24 antigen; or with HIV-1 antigen

Production of IFN-gamma; IL-12; IL-4, IL-5, IL-10, MIP1 alpha, MIP1 beta, RANTES have been evaluated with ELISA methods

P24 antigen- and HIV-1 antigen-specific IFN-gamma-producing lymphocytes have been evaluated by ELISPOT assay.

1.2.4. Statistical Analysis.

Wilcoxon rank sum test for equal medians was used. It performs a two-sided rank sum test of the hypothesis that two independent samples, in the vectors x and y, come from distributions with equal medians, and returns the p-value from the test. p is the probability of observing the given result, or one more extreme, by chance if the null hypothesis is true, i.e., the medians are equal. Small values of p cast doubt on the validity of the null hypothesis thus suggesting difference between the groups. The two sets of data are assumed to come from continuous distributions that are identical except possibly for a location shift, but are otherwise arbitrary. x and y can be different lengths.

1.3. Results

Results are presented in the set of FIGS. 9-19.

FIG. 9. Interleukin-4 production by spleen cells of female C57/BL6 mice. Upper left panel: interleukin-4 production in basal conditions (unstimulated cells). Upper right panel: interleukin-4 production by cells stimulated in vitro with HIV-1 gp160. Lower panel: interleukin-4 production by cells stimulated in vitro with HIV-1 native p24. In all panels immunization with both chimeras reduces interleukin-4 production compared to control mice (no immunization); the difference is significant for both chimeras in the upper left panel (in basal conditions), suggesting that both chimeras suppress production of IL-4 thus inhibiting activation of TH2 lymphocytes.

FIG. 10. Interleukin-10 production by spleen cells of female C57/BL6 mice. Upper left panel: interleukin-10 production in basal conditions (unstimulated cells). Upper right panel: interleukin-10 production by cells stimulated in vitro with HIV-1 gp160. Lower panel: interleukin-10 production by cells stimulated in vitro with HIV-1 native p24. Interleukin-10 production is weakly albeit significantly (upper left and lower panels) augmented in mice immunized with chimera 2 compared to control mice suggesting possible anti-inflammatory activity of chimera 2. Same effect is seen in response to IL-22 in response to HIV specific stimulation.

FIG. 11. Interleukin-5 production by spleen cells of female C57/BL6 mice. Upper left panel: interleukin-5 production in basal conditions (unstimulated cells). Upper right panel: interleukin-5 production by cells stimulated in vitro with HIV-1 gp160. Lower panel: interleukin-5 production by cells stimulated in vitro with HIV-1 native p24. Interleukin-5 production is not modified in immunized compared to control mice further confirming suppressive effect on TH2 activation.

FIG. 12. Interleukin-12 p40 subunit production by spleen cells of female C57/BL6 mice. Upper left panel: Interleukin-12 p40 subunit production in basal conditions (unstimulated cells). Upper right panel: Interleukin-12 p40 subunit production by cells stimulated in vitro with HIV-1 gp160. Lower. panel: Interleukin-12 p40 subunit by cells stimulated in vitro with HIV-1 native p24. Interleukin-12 p40 subunit production is not modified in immunized compared to control mice (only in the upper right panel mice immunized with chimera 2 show an increased production of Interleukin-12 p40 subunit). Results suggest existence of HIV specific response for chimera 2.

FIG. 13. Interleukin-12 p70 subunit production by spleen cells of female C57/BL6 mice. Upper left panel: Interleukin-12 p70 subunit production in basal conditions (unstimulated cells). Upper right panel: Interleukin-12 p70 subunit production by cells stimulated in vitro with HIV-1 gp160. Lower panel: Interleukin-12 p70 subunit by cells stimulated in vitro with HIV-1 native p24. Interleukin-12 p70 subunit production is not modified in immunized compared to control mice.

FIG. 14. RANTES production by spleen cells of female C57/BL6 mice. Upper left panel: RANTES production in basal conditions (unstimulated cells). Upper right panel: RANTES production by cells stimulated in vitro with HIV-1 gp160. Lower panel: RANTES production by cells stimulated in vitro with HIV-1 native p24. RANTES production is augmented in immunized compared to control mice (chimera 2 has a stronger effect: see upper panels). Despite not reaching statistical significance, chimera 2 has shown positive trends in stimulating HIV specific production of RANTES, suggestive of stimulatory effect on TH1 cells.

FIG. 15. MIP1-alpha production by spleen cells of female C57/BL6 mice. Upper left panel: MIP1-alpha production in basal conditions (unstimulated cells). Upper right panel: MIP1-alpha production by cells stimulated in vitro with HIV-1 gp160. Lower panel: MIP1-alpha production by cells stimulated in vitro with HIV-1 native p24. MIP1-alpha production is not modified in immunized compared to control mice. The gp160-specific response (upper right panel) is impressively increased when chimera 2 is associated with HYB2048 and IL-22 (see 3rd group of bars), suggesting that Chimera 2 activity can be significantly modified by addition cpg like molecules and IL-22 to amplify HIV specific production of MIP1-alpha and a possible direct antiviral effect of such combination.

FIG. 16. MIP1-beta production by spleen cells of female C57/BL6 mice. Upper left panel: MIP1-beta production in basal conditions (unstimulated cells). Upper right panel: MIP1-beta production by cells stimulated in vitro with HIV-1 gp160. Lower panel: MIP1-beta production by cells stimulated in vitro with HIV-1 native p24. MIP1-beta production is not modified in immunized compared to control mice.

FIG. 17. Interferon gamma production (CD4 and CD8) by spleen cells of female C57/BL6 mice. Upper left panel: Interferon gamma production (CD4 and CD8) in basal conditions (unstimulated cells). Upper right panel: Interferon gamma production (CD4 and CD8) by cells stimulated in vitro with HIV-1 gp160. Lower panel: Interferon gamma production (CD4 and CD8) by cells stimulated in vitro with HIV-1 native p24. Interferon gamma production (CD4 and CD8) is augmented in immunized compared to control mice (chimera 1 has a stronger effect: see upper right and lower panels). The gp160 response (upper right panel) is particularly impressive when chimera 1 and 2 are associated with HYB2048 and IL-22 (see 3rd and 4th groups of bars). This suggests that Chimera 2 activity can be significantly modified by addition cpg like molecules and IL-22 to amplify HIV specific production of IFN gamma stimulating cell mediated immunity in favor of TH1 balance

FIG. 18. Interferon gamma production (CD8) by spleen cells of female C57/BL6 mice. Upper left panel: Interferon gamma production (CD8) in basal conditions (unstimulated cells). Upper right panel: Interferon gamma production (CD8) by cells stimulated in vitro with HIV-1 gp160. Lower panel: Interferon gamma production (CD8) by cells stimulated in vitro with HIV-1 native p24. Interferon gamma production (CD8) is augmented in immunized compared to control mice (chimera 1 has a stronger effect: see upper left and lower panels). Data suggest that both chimeras have ability of stimulating basal and HIV specific IFN gamma production.

FIG. 19. Upper left panel: Percentage of CCR5-expressing CD4+T cells by spleen cells of female C57/BL6 mice. Upper right panel: Percentage of CXCR4-expressing CD4+T cells by spleen cells of female C57/BL6 mice. Lower panel: Ratio of CD4+/CCR5+ to CD4+/CXCR4+ cells in by spleen cells of female C57/BL6 mice.

CCR5 is reduced by both chimeras compared to non-immunized mice; CXCR4 is slightly increased by chimera 1; the ratio of CCR5 to CCR4CD4+ cells is significantly reduced by immunization with both indicating that cell susceptibility to HIV infection is significantly down modulated. The data suggest that such molecules have potential to be used as preventive vaccine or microbicide in HIV disease.

Results Show that Immune Modulation is Indeed Reached by Immunization of Mice with Chimeras.

Immune modulation associated with chimera is multifaceted as it includes an inhibitory effect on TH2 cells (reduced production of interleukin-4), an anti-inflammatory component (increased production of interleukin-10), a direct stimulation of TH1 lymphocytes as well as effect on cell mediated immunity (increased production of interleukin-12; increased number of IFNgamma-producing CD4+ and CD8+T cells).

In addition to these immunomodulatory properties, chimera reduce the susceptibility of target cells to HIV infection via two distinct mechanisms: augmented production of the soluble antiviral chemokine RANTES, and down modulation of CCR5, the main HIV co-receptor. Despite the observation that both chimera are endowed with these effects, overall immunemodulation and antiviral activities seem to be more potent in mice immunized with chimera 2. The effects of both chimera are augmented by addition of CpG-like compounds (HYB2048) and of interleukin-22. The observation that immunization with chimera results in immunemodulation and in a direct antiviral effect, suggests that chimera have potential to be used as preventive vaccine or microbicide in HIV disease.

Effect of Chimera 1 or Chimera 2 on Cell Mediated Immunity Alone and in Combination of with CCL28

It is assumed that to be successful in preventing infection mucosal vaccine against HIV needs to be capable of acting on both humoral and cell mediated immune response. Therefore, upon finding out that chimeras are capable of binding to antibodies with very high affinity, the inventors decided to examine whether chimeras were capable of affecting the cytokine production in CD4 and CD8 T cells from patients infected with HIV and whether addition of CCL28 is capable of enhancing chimera's cellular activity when used ex vivo in peripheral blood mononuclear cells (PBMCs) obtained from HIV+ patients.

CCL28 (also known as mucosae-associated epithelial chemokine, or MEC), is a recently described chemokine that is involved in the migration of IgA-secreting plasma cells and therefore may potentially play a role in the mechanism of preventing HIV infection.

CCL28 involvement in the migration of IgA-secreting plasma cells was recently shown to be correlated with specific chemokines and chemokine receptors. Thus, Ig-expressing plasma blasts and plasma cells (IgA-ASC) are characterized by a number of surface receptor proteins, including CCR9, CCR3, and CCR10, that are ligated by specific chemokines. In particular, both CCR10 and CCR3 bind CCL28.

This interaction induces the migration and the recruitment of IgA-ASC in the mucosal lamina propria. In particular, CCL28 is widely expressed and potently chemoattracs IgA-ASC originating from diverse mucosal lymphoid organs, as well as from intestinal and extraintestinal tissues, in every studied mucosal effector site both in mice and humans, including the mammary and salivary glands and the uterin and cervix mucosa.

Thus, the CCL28-CCR10/CCR3 circuit is considered to be a unifying system that plays a major role in the homing of plasmablasts and plasma cells at mucosal effector sites. Interestingly, these chemotactic abilities are limited to IgA-ASC as the migration of neither IgM or IgG ASC is stimulated by the CCL28-CCR10/CCR3 system.

The CCL28-CCR10/CCR3 circuit is also endowed with other interesting peculiarities. Thus, CCL28 has a potent antimicrobial activity directed toward both Gram-positive and Gram-negative microroganisms. Additionally, this chemokine is expressed by bone marrow stromal cells, suggesting that the ineraction of CC28 with CCR10+/CCR3+B cells may contribute to the integration between the mucosa(and the systemic immune responses.

1. Methods

CCR3/CCR10/CCL28 circuit was examined in HIV-infected patients and in HIV-exposed but uninfected individuals (ESN). CCL28 was quantified in plasma, saliva, and genital secretions of 39 HIV patients; 37 ESN; and 25 HC. CCR3, and CCR10 expression was measured in CD3+, CD19+, and CD14+ peripheral blood cells from the same individuals. CCL28 was also quantified in breast milk from 65 HIV-infected women and 9 uninfected controls.

Results are summarized as follows:

1. The concentration of CCL28 is increased in plasma, saliva, and genital secretions of ESN and HIV patients compared to HC.

2. The percentage and the mean fluorescence intensity (MFI), a relative measure of the surface density on a cellular level of CCR3 and CCR10 on CD19+/cells is increased in ESN and HIV patients compared to HC.

3. Positive correlations are observed in plasma, saliva, and genital secretions of HIV and ESN between CCL28 and HIV-specific IgA.

ESN iare characterized by the presence of high concentrations of HIV-specific IgA, discovering that CCL28 plays important role in biochemical cascade of events preventing infection in HIV negative exposed individuals (ESN), this rendering this molecule as a possible target for antiviral (HIV) vaccine.

The results suggest that CCL28 is important for healthy antigen specific immune response to infections, namely viruses as evidenced by data on HIV. It is likely that this molecule can be used alone or as an adjuvant for antiviral (HIV) vaccine.

2. Methods

To evaluate a possible effect of CCL28 on cell mediated immunity, peripheral blood mononuclear cells of HIV-infected individuals were cultured in the presence of CCL28-expressing plasmids or/and in presence of the two chimeric proteins.

2.1. Cytokine Production by Antigen and Mitogen-Stimulated T Lymphocytes.

Intracellular Analyses

PBMC are resuspended in medium supplemented with 2% AB+ serum and sowed in sterile tubes with supernatant of insect cells (control), Chimera 1 (0.5 μg), Chimera 2 (0.05 μg), Chimera 1 or 2+ pCpGCCL28, (murine CCL28 cloned in the pCpG expression vector from invitrogen), ENV (a pool of five synthetic peptides from gp160 of HIV-1 at 5 μM final concentration), GAG (a pool of six synthetic peptides from gag p17 and gag p24 (GAG) and staphylococcal enterotoxin B (SEB, 40 μg/ml) (Sigma, St Louis, Mo.)(positivecontrol). Antibody to CD28 (R&D Systems, Minneapolis, Minn.) is added during incubation at a dose of 1 μg/well to facilitate co-stimulation. Brefeldin A (Sigma Aldrich) (10 μg/ml final oncentration) is added to each tube. PBMC are incubated for 42 hours and then stained for CD4/IFN-gamma/IL-2 and for CD8/IFN-gamma/TNF-alfa usinf the IntraPrep Kit (Beckman Coulter).

2.2. Proliferation Assay with CFSE and Phenotyping of Proliferating Cells

The CFSE Flow method provides a simple and sensitive technique for multiple parameter analysis of cells. This method permits the study of specific populations of proliferating cells. PBMC are first incubated with membrane permeable, non-fluorescent CFSE which passively diffuses into cells. Excess dye is washed away and PBMC are induced to proliferate by in vitro stimulation with supernatant of insect cells (control), Chimera 1 (0.05 μg), Chimera 2 (0.05 μg), Chimera 1 or 2+pCpGCCL28, (murine CCL28 cloned in the pCpG expression vector from invitrogen), ENV (a pool of five synthetic peptides from gp160 of HIV-1 at 5 μM final concentration), GAG (a pool of six synthetic peptides from gag p17 and gag p24 (GAG) and staphylococcal enterotoxin B (SEB, 40 μg/ml) (Sigma, St Louis, Mo.)(positivecontrol). The cells are maintained in culture for five days. Staining with fluorescence labeled antibodies for cell surface molecules CD4 AND CD8 allows the examination of the proliferation of specific lymphocyte subsets.

3. Results

FIGS. 20 and 21 show respectively the effect of Chimera 1 (CH1) and chimera 2 (CH2)+/−CCL28 on the production of IL-2 and IFN-gamma by CD4 and on the production of IFN-gamma and TNF-alpha by CD8 T cells.

No effect of Chimera 1 (0.5 μg)+/−CCL28 on production of cytokines by CD4 or by CD8 T cells was observed (FIGS. 22 and 23). Chimera 2 alone can increase slightly the production of IL-2 by CD4 T cells from 5.2% in the medium to 7.2% (FIG. 20). CCL28-expressing plasmids together with gp120/CD4 chimera 2 can have an extremely potent effect on CD4+, IL-2-secreting T cells. Indeed, the addition of CCL28 to Chimera 2 increased the CD4+T cells expressing IL-2 from 5.2% to 15.6% (FIG. 20). Similar results have been shown on CD8 T cells expressing TNF-alpha for which the addition of CCL28 to Chimera 2 increased the CD8+T cells expressing TNF-alpha from 4.1% to 12.2(FIG. 21).

In conclusion, the addition of CCL28 increase the immunogenicity to Chimera 2.

These results represent a pivotal finding for a novel strategy of eliciting humoral and cell mediated immunity in infectious diseases aimed at mucosal immunity.

FIGS. 22 and 23 show the effect of Chimera 1 (CH1) and chimera 2 (CH2)+/−CCL28 on CD4 T cell proliferation (FIG. 22) and CD8 T cell proliferation (FIG. 23) using the CFSE method. Chimera 1 and 2 with or without CCL28 induces a strong proliferation of CD4 T cells (FIGS. 22 A and B). Indeed a 2-3 fold increased proliferation was observed compared to the control with insect cell supernatant (FIG. 22B). The same results have been observed with CD8 bright T cell but to a lower extent (FIG. 23) as the proliferation of CD8 T cells is increased from 0.4% to 1-2%.

These results suggest that Chimera 1 and 2 with or without CCL28 induce a strong proliferation of CD4 T cells and to a much lower extend of CD8bright T cells.

The invention thus provides chimeras which, in addition to having high affinity to anti HIV antibodies, also have potent immunomodulatory and antviral effect that can be amplified in combination with IL-22 & CCL28 to form a potent and a novel product for vaccine/microbicide.

1. Chimeric HIV-1 gp120 glycoproteins, wherein at least a part of gp120 variable region V1 and/or V2 is replaced by a CD4-derived sequence to obtain the exposition of CD4 induced epitopes or CD4i capable of inducing a specific humoral immune response.

2. The chimeric HIV-1 gp120 glycoproteins of claim 1, wherein the CD4-derived sequence replaces V1 and a part of V2 gp120 variable regions.

3. The chimeric HIV-1 gp120 glycoproteins of claim 1, wherein the CD4-derived peptide mimics the CDR2-like loop of human CD4 receptor.

4. The chimeric HIV-1 gp120 glycoproteins of claim 1, which comprises 15 to 35 amino acids.

5. The chimeric HIV-1 gp120 glycoproteins of claim 4, which comprises 20 to 30 amino acids.

6. The chimeric HIV-1 gp120 glycoproteins of claim 1, wherein said peptide has 28 amino acids.

7. The chimeric HIV-1 gp120 glycoproteins of claim 6, having sequence SEQ ID NO 5 CNLEACQKRCQSLGLQGKCAGSFCAC.

8. The chimeric HIV-1 gp120 glycoproteins of claim 2, wherein the CD4-derived sequence replaces V1 and 10 to 20 amino acids from V2 loop, particularly 16 amino acids.

9. The chimeric HIV-1 gp120 glycoproteins of claim 1, which are recognized by anti-CD4i monoclonal antibodies, but are not recognized by anti-CD4 antibodies.

10. The chimeric HIV-1 gp120 glycoproteins of claim 1, characterized in that they bind CD4 receptor and CCR5 co-receptor.

11. The chimeric HIV-1 gp120 glycoproteins of FIGS. 1B and 1C.

12. The chimeric HIV-1 gp120 glycoproteins of claim 1 in combination with TLR ligands.

13. The chimeric HIV-1 gp120 glycoproteins of claim 1 in combination with IL22 and/or CCL28.

14. Antisera containing polyclonal antibodies directed to CD4i epitopes such as exposed in the chimeric HIV-1 gp120 glycoproteins of claim 1.

15. Polyclonal antibodies directed to CD4i epitopes such as exposed in the chimeric HIV-1 gp120 glycoproteins of claim 1.

16. Monoclonal antibodies directed to CD4i epitopes such as exposed in the chimeric HIV-1 gp120 glycoproteins of claim 1.

17. The DNA constructs encoding the chimeric HIV-1 gp120 glycoproteins of claim 1.

18. Expression vectors comprising a DNA construct according to claim 17.

19. Host cells transfected by the vectors of claim 18.

20. A vaccine composition specific to HIV-1 infections, comprising an effective amount of at least one antigenic chimeric HIV-1 constructs according to claim 1, with a carrier, or a combination thereof with TLR ligands.

21. The vaccine composition of claim 20 for preventing HIV-1 infections.

22. A pharmaceutical composition for alleviating and/or preventing and/or treating an HIV-1 infection comprising an effective amount of at least one chimeric HIV-1 gp120 glycoproteins according to claim 1, or a combination thereof with TLR ligands, IL22 and/or CCL28.
